(19) United States
(12) Patent Application Publication
(10) Pub. No.: US 2018/0126857 A1
(43) Pub. Date: May 10, 2018

(54) ELECTRIC VEHICLE POWERED BY CAPACITIVE ENERGY STORAGE MODULES

(71) Applicant: Capacitor Sciences Incorporated, Menlo Park, CA (US)

(72) Inventor: Ian S. G. Kelly-Morgan, Alameda, CA (US)

(21) Appl. No.: 15/675,614

(22) Filed: Aug. 11, 2017

Related U.S. Application Data
(63) Continuation-in-part of application No. 15/430,307, filed on Feb. 10, 2017.
Application No. 15/430,307 is a continuation-in-part of application No. 15/043,186, filed on Feb. 12, 2016.
Application No. 15/043,186 is a continuation-in-part of application No. 15/043,209, filed on Feb. 12, 2016.
Application No. 15/043,209 is a continuation-in-part of application No. 15/043,247, filed on Feb. 12, 2016.
Application No. 15/043,247 is a continuation-in-part of application No. 15/043,315, filed on Feb. 12, 2016.
Application No. 15/430,307 is a continuation-in-part of application No. 15/043,186, filed on Feb. 12, 2016.
Application No. 15/043,186 is a continuation-in-part of application No. 15/043,209, filed on Feb. 12, 2016.
Application No. 15/043,209 is a continuation-in-part of application No. 15/043,247, filed on Feb. 12, 2016.
Application No. 15/043,247 is a continuation-in-part of application No. 15/043,315, filed on Feb. 12, 2016.
(60) Provisional application No. 62/294,949, filed on Feb. 12, 2016.

(51) Int. Cl. B60L 11/18; B60K 6/28; B60K 6/46; B60K 6/48; H01G 4/14; H01G 4/005; H01G 4/32; C08F 220/36; C08F 220/06; C08F 220/18; C08F 220/34; C08L 33/02; C08L 33/10; C08L 33/14; C07D 471/06

(52) U.S. Cl. CPC B60L 11/1811; B60K 2001/0438; B60K 6/46; B60K 6/48; H01G 4/14; H01G 4/005; H01G 4/32; C08F 220/36; C08F 220/06; C08F 220/18; C08F 220/34; C08L 33/02; C08L 33/10; C08L 33/14; C07D 471/06; Y10S 903/907; B60Y 2200/91; B60Y 2200/92; B60Y 2400/114; B60Y 2400/61; B60K 6/28

(57) ABSTRACT

The invention relates to a capacitor based energy storage module and an electric vehicle using the same. For providing an electric vehicle which can meet the requirements of long distance running and rapid acceleration at the same time, the electric vehicle of present invention comprising: An electric control unit controlling all operations of the electric vehicle; An energy storage unit having one or more energy devices based on meta-capacitor which provides needed electric energy of the electric vehicle; A DC-DC converter receiving control signal from said electric control unit to convert the energy from said energy storage unit then provide to motor; A motor converting the electric energy into mechanical energy to drive the wheels; Wheels are driven by the motor to make the electric vehicle run.

### CLAIM OF PRIORITY

This application is a continuation-in-part of U.S. patent application Ser. No. 15/430,307 filed Feb. 10, 2017 which is hereby incorporated herein by reference in its entirety. U.S. patent application Ser. No. 15/430,307 claims the benefit of U.S. Provisional Application No. 62/294,949 filed Feb. 12, 2016, which is hereby incorporated herein by reference in its entirety. This application is a continuation-in-part of U.S. patent applications Ser. Nos. 15/043,315, 15/043,186, 15/043,209, and 15/043,247, all of which were filed Feb. 12, 2016, the entire contents of all of which are incorporated herein by reference.

### BACKGROUND

### 1. Field of the Disclosure

The present disclosure relates to an electric vehicle which runs by means of a motor using a capacitive energy storage module (CESM) which can provide the energy for long distance travel and emergency acceleration.

### 2. Description of the Related Art

It is known that an electric vehicle using a battery as a power source requires significant time to recharge due to the physical and chemical properties of batteries (e.g. internal resistance), and that complicated electronic arrangements and systems are required to deliver both accelerating power and long travel ranges on a single charge. Additionally, existing battery technology employs advanced primary and secondary battery architecture or hybrid capacitor-battery systems to achieve accelerating power, extended single charge travel distances, and improve cycle lifetime of the power pack. For these reason, it is desirable to use a capacitive energy storage module capable of high power density and high energy density.

In existing pure electric vehicles, use one or more electricity power supplies, for example, a first source of stored electrical energy may be used to provide longer-lasting energy while a second source of stored electrical energy may be used, for example, to provide higher-power for acceleration.

Hybrid electric vehicles may combine an internal combustion engine and an electric motor powered by an energy storage device, such as a lithium battery pack mated with a traction motor, to propel the vehicle. Such a combination may increase overall fuel efficiency by enabling the combustion engine and the electric motor to each operate in respective ranges of increased efficiency. Electric motors, for example, may be efficient at accelerating from a standing start, while combustion engines may be efficient during sustained periods of constant engine operation, such as in highway driving. Most hybrid electric vehicles have an electric motor to boost initial acceleration allowing combustion engines in hybrid vehicles to be smaller and more fuel efficient.

One of the biggest problems of existing electric vehicles is shortage of range, limited to the battery capacity, most pure electric vehicles have a range of 100-200 km, and a few models are offered with ranges between about 350-540 kilometers (km). In addition, present electric vehicles suffer from the slow recharging rate of batteries. Electric vehicles generally require several hours to recharge, and the best chargers still require tens of minutes to recharge long range battery packs above 50% charge. Further complicating recharging battery packs are issues of power density, internal resistance, and thermal management systems to prevent thermal runaway in lithium cells. These interrelated challenges also conspire to reduce the lifespan of a battery pack, which is a tradeoff engineers balance with the convenience of so-called rapid charging. Alternative arrangements may provide for using higher voltages to reduce charging time. But, high voltage charging can significantly reduce the lifespan of batteries.

In order for an EV to match the performance specifications of a small internal combustion engine (ICE) vehicle (i.e. a maximum speed of 120 km/h, a 150 km range per charge, and 0-40 km/h acceleration within 4 seconds), an energy storage device must have a specific energy greater than 75 Watt-hours per kilogram (Wh/kg), an energy density greater than 127 Watt-hours per liter (Wh/L), a power output greater than 160 Watts per kilogram (W/kg). To begin to be competitive with ICE vehicles, the lifespan of the energy storage device needs to exceed 1000 charge-discharge cycles with less than a 40% decay of charge capacity over the lifespan. According to www.EMValley.com's Battery Status Tracker tool, current lithium batteries (as of Aug. 9, 2017) have a specific energy topping out at 269 Wh/kg and battery packs top out at 156 Wh/kg. Further, materials, form factor, and other packaging parameters clearly have an impact on the specific energy. The bottleneck of electric vehicle development lies in the energy storage device. Examples of the trend for specific energy for current lithium batteries used in electric vehicles are shown in FIGS. 12A-12B and the corresponding trend for EPA ranges for current electric vehicles are shown in FIG. 12C.

Electric power rating and design, chemistry, and thermal management of battery packs not only determine the recharge rate of the battery pack; but also determine the efficiency of regenerative braking. According to K. Smith et al. (“Power and Thermal Characterization of a Lithium-Ion Battery Pack For Hybrid-Electric Vehicles,” J. Power Sources 160, 662 (2006)), battery type and operating temperature effects the braking power and thus regenerative braking power of an EV or Hybrid vehicle.

A typical NRC18650 lithium ion battery has a specific energy greater than 225 Wh/kg and a specific power of about 0.233 kW/kg. Additionally, an example 3.0 volt/3000 F supercapacitor has a specific energy of about 7.2 Wh/kg and a specific power of about 7.7 kW/kg. A power battery pack of NRC18650 lithium ion batteries (LIB) needs to be configured and about 30 times heavier than a supercapacitor to generate an equivalent power output of a supercapacitor. Conversely, a power pack of the 3.0 V/3000 F supercapacitor (supercap) would need to be more or less 30 times heavier than a LIB pack to store an equivalent amount of energy.

It is an advancement in the art for an EV or hybrid EV to have an energy storage system that includes rapid charging, highly efficient regenerative braking, and comprised of cells with specific energy greater than 1000 Wh/kg and a specific power greater than 8 kW/kg. The advantages of which are a lightweight energy storage device with a high-power rating that enables efficient regenerative braking, fast acceleration, and rapid charging without compromising single charge travel range. Further, it is an advance to store 1000 Wh/kg in an electric device that does not have the safety issues and thermal constraints of an electro-chemical cell. Additionally, it is an advance to extend the lifespan of an EV's or hybrid EV's electric energy storage device without significant decay of storage capacity due to deep cycling effects (e.g. thermal and electro-chemical degradation).

### SUMMARY

One of the purposes of the present invention is to provide said electric vehicle, which is able to meet the requirements of long distance travelling, at mean time can meet the requirement of rapid accelerating, and other requirements described above. In order to meet the purpose, another purpose of the present invention is to provide an energy storage device, which has short charging time, plenty of capacity, light weight, and is safe and reliable.

To realize the purposes, one embodiment of present invention is an electric vehicle, comprising, an electric control unit controlling the whole operations of said electric vehicle, an energy storage unit having an energy device based on metacapacitor which provides needed electric energy of said electric vehicle, a DC-DC converter receiving control signal from said electric control unit to converter the energy from said energy storage unit then provide to motor, and a motor converter the electric energy into mechanical energy to drive the wheels. Wheels are driven by said motor to make said electric vehicle run.

An electric vehicle of the present invention in one aspect provides a CESM comprising more than one individual CESCs having anodes and cathodes and an interconnection system, wherein the interconnection system connects the anodes and cathodes of the individual CESCs to create a common anode and common cathode of the capacitive energy storage module.

In another aspect, the present invention provides a capacitive energy storage system comprising one or more capacitive energy storage modules, an interconnection system and a system control computer.

Additionally, an electric vehicle of the present invention comprises a frame structure including a pair of right and left side members made of metal, arranged at a lower part of a vehicle body, a floor panel made of metal provided on the frame structure, a CESM including a CESM case arranged between the pair of side members on the undersurface side of the floor panel, the CESM case containing therein CESM cells and electric components electrically connected to the CESM cells, and the upper side of the electric components being optionally electromagnetically shielded by the floor panel, a front electromagnetic shield portion arranged on the vehicle-front side with respect to the electric components, a rear electromagnetic shield portion arranged on the vehicle-rear side with respect to the electric components, and an under electromagnetic shield portion arranged on the undersurface side of the CESM case.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate embodiments of the invention, and together With the general description given above and the detailed description of the embodiments given below, serve to explain the principles of the invention.

FIG. 1 is a perspective view of an electric vehicle, CESM, and under cover according to an embodiment of the present invention.

FIG. 2 is a perspective view of the frame structure and CESM of the electric vehicle shown in FIG. 1.

FIG. 3 is a perspective view of the CESM case of the CESM and beam members shown in FIG. 2.

FIG. 4 is a side view of the frame structure and CESM of the electric vehicle shown in FIG. 1.

FIG. 5 is a plan view of the frame structure and CESM of the electric vehicle shown in FIG. 1 viewed from above.

FIG. 6 is a perspective view of the frame structure and under cover of the electric vehicle shown in FIG. 1 viewed from below.

FIG. 7 is a perspective view of insert members to be embedded in the tray member of the CESM case shown in FIG. 2.

FIG. 8 shows an example of a capacitive energy storage module having two or more networked energy storage cells according to an alternative aspect of the present disclosure.

FIG. 9 schematically shows an energy storage cell according to aspects of the present disclosure.

FIG. 10A schematically shows a meta-capacitor with flat and planar electrodes according to aspects of the present disclosure.

FIG. 10B schematically shows a meta-capacitor with rolled (circular) electrodes according to aspects of the present disclosure.

FIG. 11A is a schematic diagram of an electric vehicle power system that may be used in conjunction with aspects of the present disclosure

FIG. 11B is a schematic diagram of a series hybrid vehicle power system that may be used in conjunction with aspects of the present disclosure.

FIG. 11C is a schematic diagram of a parallel hybrid vehicle power system that may be used in conjunction with aspects of the present disclosure.

FIGS. 12A-12B are graphs showing the trend for specific energy for current lithium batteries used in electric vehicles.

FIG. 12C is a graph showing the trend for EPA ranges for current electric vehicles.

### DETAILED DESCRIPTION

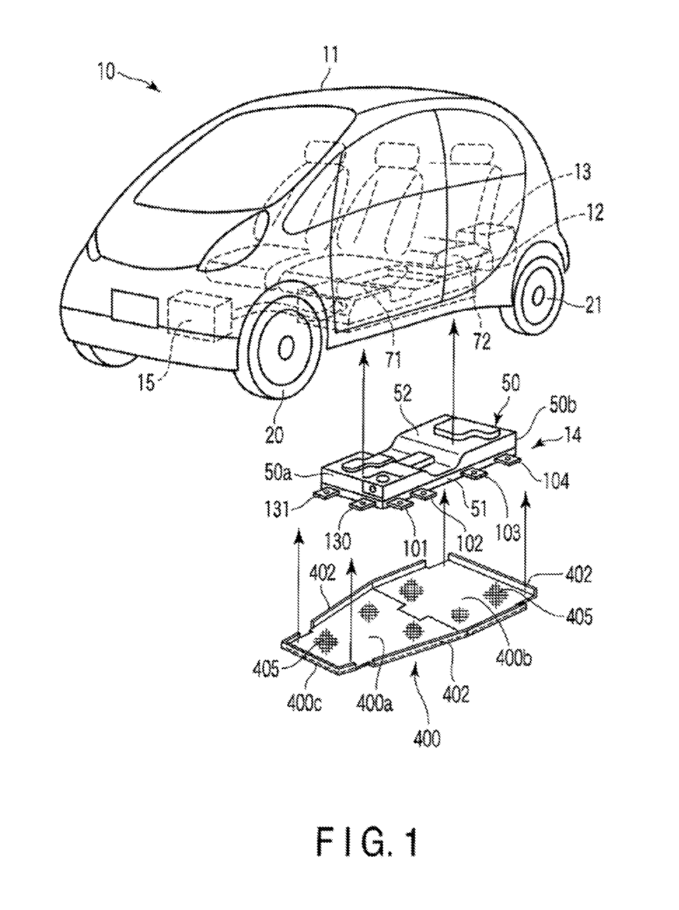

Aspects of the Present Disclosure are Described below with Reference to FIGS. 1 to 11C.

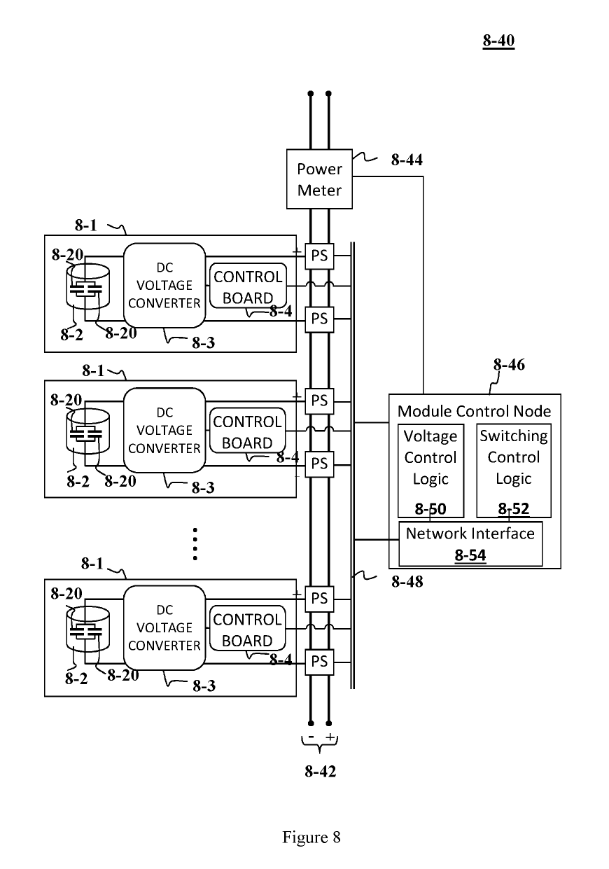

In another aspect of the present disclosure, a capacitive energy storage module 8-40, e.g., as illustrated in FIG. 8. In the illustrated example, the energy storage module 8-40 includes two or more energy storage cells 8-1 of the type described above. Each energy storage cell includes a capacitive energy storage device 8-2 having one or more metacapacitors 8-20 and a DC-voltage converter 8-3, which may be a buck converter, boost converter, or buck/boost converter. In addition, each module may include a control board 8-4 and an (optional) cooling mechanism (not shown). By way of example, and not by way of limitation, the capacitive energy storage module 8-40 may include a cooling mechanism in thermal contact with the capacitive energy storage device and/or the DC-voltage converter. Such a cooling mechanism may be, e.g., a passive cooling mechanism, an active cooling system using air, water, ethylene glycol as a coolant; phase-change material, or any combination thereof. In some implementations, the cooling mechanism may include a reservoir containing a solid to liquid phase change material, such as paraffin wax. The module 8-40 may further include an interconnection system that connects the anodes and cathodes of the individual energy storage cells to create a common anode and common cathode of the capacitive energy storage module.

In some implementations, the control board 8-4 may include a programmable electronic device configured so that one or more energy storage cells 8-1 can supply a constant output voltage that is programmable by the control board.

In yet another aspect, some implementations, the interconnection system includes a parameter bus 8-42 and power switches PSW. Each energy storage cell 8-1 in the module 8-40 may be coupled to the parameter bus 8-42 via the power switches PSW. These switches allow two or more modules to be electrically coupled in parallel or in series via two or more rails that can serve as the common anode and common cathode. The power switches can also allow one or more energy storage cells to be disconnected from the module, e.g., to allow for redundancy and/or maintenance of cells without interrupting operation of the module. The power switches PSW may be based on solid state power switching technology or may be implemented by electromechanical switches (e.g., relays) or some combination of the two.

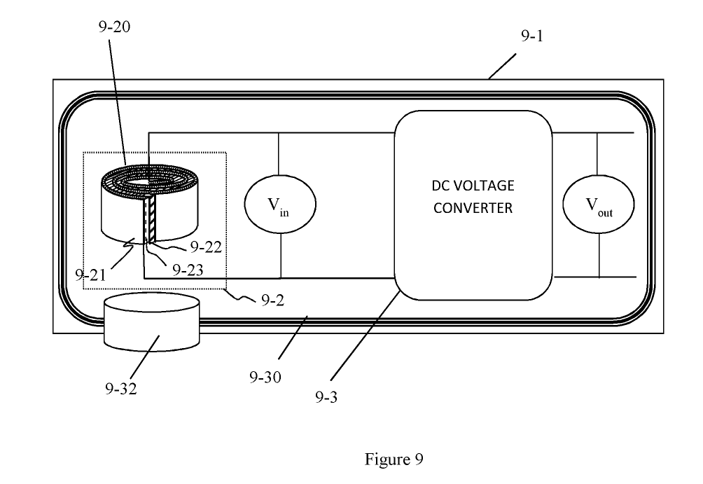

The present disclosure provides a capacitive energy storage cell (CESC) comprising one or more capacitive energy storage device (CESD) and a DC-voltage conversion device. FIG. 9 schematically shows an example of a CESD 9-1 comprising a metacapacitor 9-2 electrically connected to a DC-voltage conversion device 9-3. In one implementation CESD 9-1, metacapacitor 9-2 is two electrodes and metadielectric material layer 9-23 in the form of long strips of material that are sandwiched together and wound into a coil along with an insulating material, e.g., a plastic film such as polypropylene or polyester to prevent electrical shorting between the electrodes 9-21, 9-22. In some implementations, the DC-voltage conversion device 9-3 may include a buck converter, boost converter, buck/boost converter, bi-directional buck/boost (split-pi) converter, auk converter, single-ended primary inductor converter (SEPIC), inverting buck/boost converter, or four-switch buck/boost converter or some combination of two or more of these. In some implementations, the DC-voltage converter may include power electronics switches based on a material such as silicon (Si) insulated-gate bipolar transistors (IGBTs), silicon carbide (SiC), or metal oxide. In some implementations, the DC-voltage converter may include power electronics switches; and said power electronics switches comprise multiple switch elements stacked in series.

Examples of said CESM, CESC, and CESD are described in detail in commonly-assigned U.S. patent application Ser. No. 15/043,315 (Attorney Docket No. CSI-024), filed Feb. 12, 2016, the entire contents of which are incorporated herein by reference.

Aspects of the present disclosure are directed to first time use of metacapacitors for powering vehicles. Advantages are that metacapacitors have higher energy density and power density that current methods used to achieve this. Said metadielectric materials are comprised of composite molecules having supra-structures formed from polymers. Non-limiting examples of said polymers include so-called YanLi polymers, so-called Sharp polymers, so-called non-linear static dielectric materials, and so-called electro-polarizable compounds, so-called Furuta co-polymers and so-called para-Furuta polymers as described in detail in commonly-assigned U.S. patent application Ser. No. 15/449,587 (Attorney Docket No. CSI-050B-US) filed Mar. 3., 2017, Ser. No. 15/469,126 (Attorney Docket No. CSI-050) filed Mar. 24, 2017, Ser. No. 15/090,509 (Attorney Docket No. CSI-051) filed Apr. 4, 2016; Ser. No. 15/043,247 (Attorney Docket No. CSI-046) filed Feb. 12, 2016; and Ser. No. 15/043,186 (Attorney Docket No. CSI-019A) filed Feb. 12, 2016; and Ser. No. 15/043,209 (Attorney Docket No. CSI-019B) filed Feb. 12, 2016; respectively, all filed Feb. 12, 2016, the entire contents of which are incorporated herein by reference. Aspects of the present disclosure include implementations in which the metadielectric material layer includes one or more types of Sharp polymers and/or one or more types of Furuta polymers. As used herein, a metacapacitor is a dielectric film capacitor whose dielectric film is a metadielectric material, which is disposed between a first electrode and second electrode. In one embodiment, said electrodes are flat and planar and positioned parallel to each other. In another embodiment, the metacapacitor comprises two rolled metal electrodes positioned parallel to each other. Additionally, a metadielectric material comprises of YanLi polymers, non-linear static dielectric materials, electro-polarizable compounds, Sharp polymers, Furuta polymers, and/or a mixture thereof.

YanLi polymer are polymers comprised of a polarizable core side chain and an electrically resistive side chain (e.g. branched and unbranched hydrocarbons and halocarbons with no extend no extend pi-conjugated fragments).

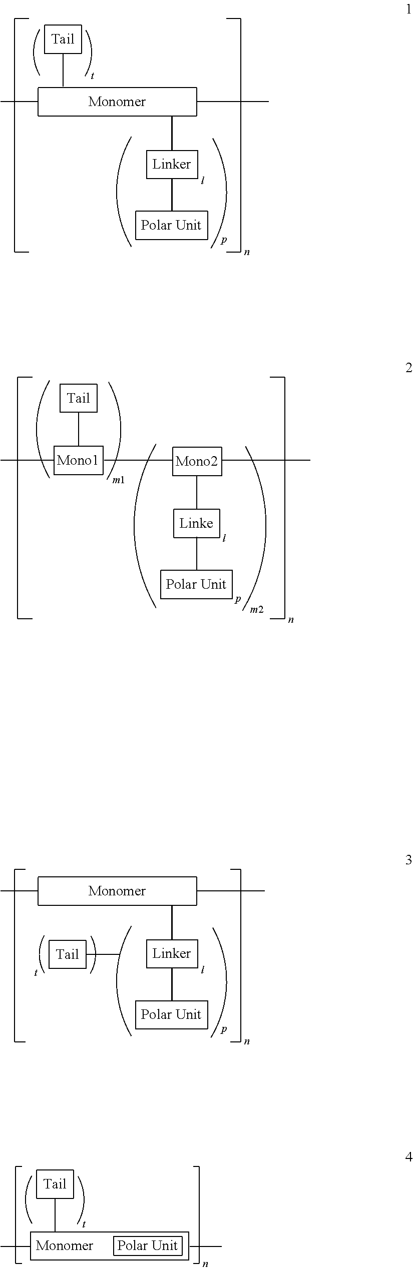

wherein “Monomer”, Mono 1 and Mono2 each represent a backbone unit; “Tail” is an electrically resistive side chain; “Polar Unit” is a polarizable core; “Linker” is a handle or a connecting group; “m1”, “m2”, “p” and “t” represent an integer indicating the number of occurrences of monol, mono2, Polar Unit, and Tail respectively; and “1” is equal to 0 or 1; “n” is an integer greater than or equal to 1 representing the number of repeat units of the composite oligomeric material.

### EXAMPLE 1

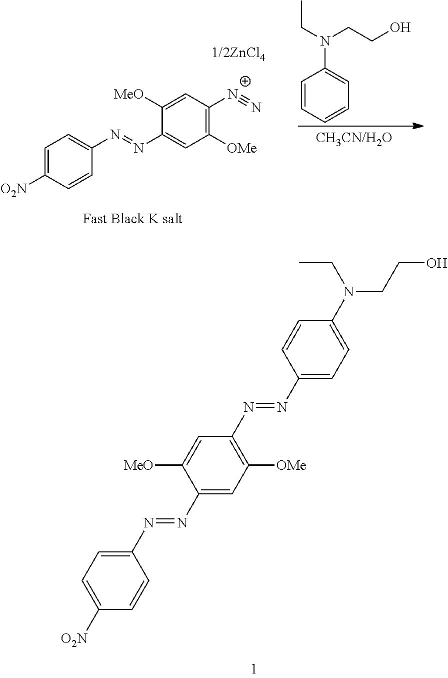 [SMILES: COc1cc([N+]#N)c(OC)cc1/N=N/c1ccc([N+](=O)[O-])cc1 ; CCN(CCO)c1ccccc1 ; CCN(CCO)c1ccc(/N=N/c2cc(OC)c(/N=N/c3ccc([N+](=O)[O-])cc3)cc2OC)cc1]

2-((4-((E)-(2,5-dimethoxy-4-((E)-(4-nitrophenyl) diacetyl)phenyl) diacetyl)phenyl)(ethyl) amino)ethan-1-ol (1). Fast Black K Salt (25%, 30 g) was dissolved in 250 mL acetonitrile and 250 mL NaOAc buffer solution (pH=4) and the resulting solution was stirred for 1 hour and then sonicated for 15 min, followed by vacuum filtration. The filtrate was dropwise added to a solution of 2-(ethyl(phenyl)amino)ethan-1-ol (4.1 g in 65 mL acetonitrile) at 0° C. The resultant solution was stirred at room temperature for 16 hours and the precipitate was filtered out and washed with mix solvent of acetonitrile/water (1:1) and dried under vacuum. The product was obtained as a black powder.

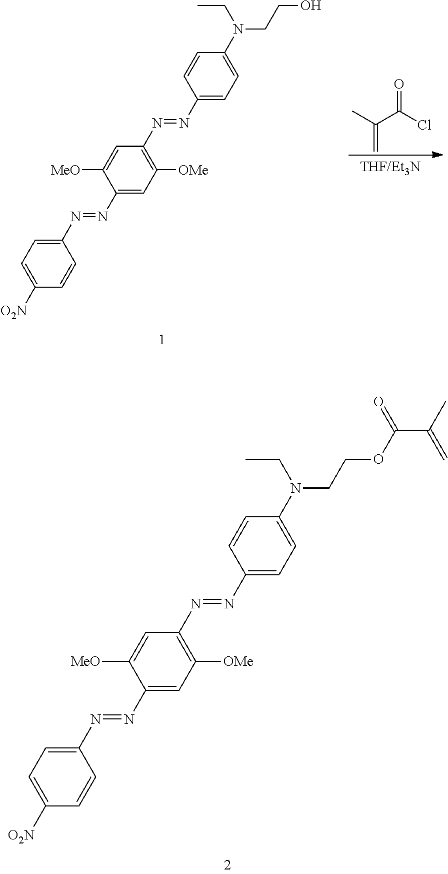

2-((4-((E)-(2,5-dimethoxy-4-((E)-(4-nitrophenyl)diazenyl)phenyl)diazenyl)phenyl)(ethyl) amino)ethyl methacrylate (2). To the solution of compound 1 (5.0 g) and triethylamine (4.4 mL) in 70 mL THF (anhydrous) at 0° C., was dropwise added a solution of methacryloyl chloride (3.1 mL) in THF (anhydrous, 10 mL). The resulting solution was warmed up to room temperature and was stirred overnight at room temperature. The reaction solution was filtered and THF was used to wash the insoluble; the filtrate was concentrated under vacuum and diluted in dichloromethane. The diluted solution was washed with water and the solvent was removed under vacuum. The crude product was purified with column chromatography and 3.2 g pure product was isolated as a black powder.

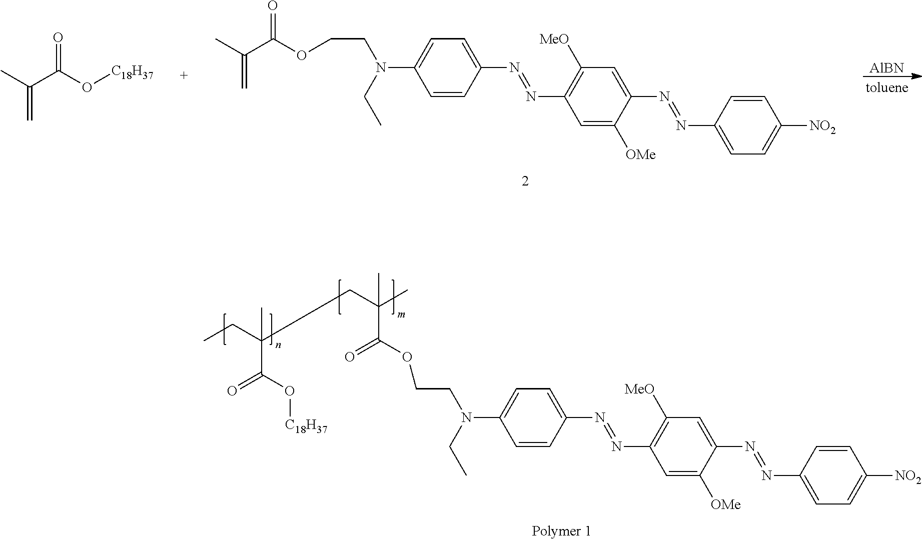

Polymer 1. Compound 2 (2.0 g), stearylmethacrylate (1.2 g) and AIBN (160 mg) were dissolved in anhydrous toluene (12 mL) in a sealed flask and the resulting solution was heated to 85° C. for 18 hours and then cooled to room temperature. The polymer was obtained by precipitating in isopropanol.

Sharp polymers are composites of a polarizable core inside an envelope of hydrocarbon (saturated and/or unsaturated), fluorocarbon, chlorocarbon, siloxane, and/or polyethylene glycol as linear or branched chain oligomers covalently bonded to the polarizable core that act to insulate the polarizable cores from each other, which favorably allows discrete polarization of the cores with limited or no dissipation of the polarization moments in the cores. The polarizable core has hyperelectronic or ionic type polarizability. “Hyperelectronic polarization may be viewed as the electrical polarization in external fields due to the pliant interaction with the charge pairs of excitons, in which the charges are molecularly separated and range over molecularly limited domains.” (See Roger D. Hartman and Herbert A. Pohl, “Hyper-electronic Polarization in Macromolecular Solids”, Journal of Polymer Science: Part A-1 Vol. 6, pp. 1135-1152 (1968)). Ionic type polarization can be achieved by limited mobility of ionic parts of the core molecular fragment.

A Sharp polymer has a general structural formula:

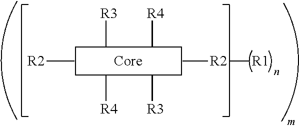

Where Core is an aromatic polycyclic conjugated molecule comprising rylene fragments. This molecule has flat anisometric form and self-assembles by pi-pi stacking in a column-like supramolecule. The substitute R1 provides solubility of the organic compound in a solvent. The parameter n is number of substitutes R1, which is equal to 0, 1, 2, 3, 4, 5, 6, 7 or 8. The substitute R2 is an electrically resistive substitute located in terminal positions, which provides resistivity to electric current and comprises hydrocarbon (saturated and/or unsaturated), fluorocarbon, siloxane, and/or polyethyleneglycol as linear or branched chains. The substitutes R3 and R4 are substitutes located on side (lateral) positions (terminal and/or bay positions) comprising one or more ionic groups from a class of ionic compounds that are used in ionic liquids connected to the aromatic polycyclic conjugated molecule (Core), either directly, e.g., with direct bound SP2-SP3 carbons, or via a connecting group. The parameter m is a number of the aromatic polycyclic conjugated molecules in the column-like supramolecule, which is in a range from 3 to 100,000.

In another embodiment of the composite organic compound, the aromatic polycyclic conjugated molecule comprises an electro-conductive oligomer, such as a phenylene, thiophene, or polyacene quinine radical oligomer or combinations of two or more of these. In yet another embodiment of the composite organic compound, the electro-conductive oligomer is selected from phenylene, thiophene, or substituted and/or unsubstituted polyacene quinine radical oligomer of lengths ranging from 2 to 12 or combination of two or more of these. Wherein the substitutions of ring hydrogens by O, S or NR5, and R5 is selected from the group consisting of unsubstituted or substituted C₁-C₁₈alkyl, unsubstituted or substituted C₂-C₁₈alkenyl, unsubstituted or substituted C₂-C₁₈alkynyl, and unsubstituted or substituted C₄-C₁₈ aryl.

In some embodiments, the substitute providing solubility (R1) of the composite organic compound is C_{X}Q_{2X+1}, where X≥1 and Q is hydrogen (H), fluorine (F), or chlorine (Cl). In still another embodiment of the composite organic compound, the substitute providing solubility (R1) of the composite organic compound is independently selected from alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, butyl, iso-butyl and tent-butyl groups, and the aryl group is selected from phenyl, benzyl and naphthyl groups or siloxane, and/or polyethylene glycol as linear or branched chains.

In some embodiments, at least one electrically resistive substitute (R2) of the composite organic compound is C_{X}Q_{2X+1}, where X≥1 and Q is hydrogen (H), fluorine (F), or chlorine (Cl). In another embodiment of the composite organic compound, at least one electrically resistive substitute (R2) is selected from the list comprising —(CH₂)ₙ—CH₃, —CH((CH₂)ₙCH₃)₂) (where n≥1), alkyl, aryl, substituted alkyl, substituted aryl, branched alkyl, branched aryl, and any combination thereof and wherein the alkyl group is selected from methyl, ethyl, propyl, butyl, iso-butyl and tent-butyl groups, and the aryl group is selected from phenyl, benzyl and naphthyl groups. In yet another embodiment of the composite organic compound.

In some embodiments, the substitute R1 and/or R2 is connected to the aromatic polycyclic conjugated molecule (Core) via at least one connecting group. The at least one connecting group may be selected from the list comprising the following structures: ether, amine, ester, amide, substituted amide, alkenyl, alkynyl, sulfonyl, sulfonate, sulfonamide, or substituted sulfonamide.

In some embodiments, the substitute R3 and/or R4 may be connected to the aromatic polycyclic conjugated molecule (Core) via at least one connecting group. The at least one connecting group may be selected from the list comprising CH₂, CF₂, SiR₂O, CH₂CH₂O, wherein R is selected from the list comprising H, alkyl, and fluorine. In another embodiment of the composite organic compound, the one or more ionic groups include at least one ionic group selected from the list comprising [NR₄]⁺, [PR₄]⁺ as cation and [—CO₂]⁻, [—SO₃]⁻, [—SR₅]⁻, [—PO₃R]⁻, [—PR₅]⁻ as anion, wherein R is selected from the list comprising H, alkyl, and fluorine.

In some implementations, the aromatic polycyclic conjugated molecule (Core) comprises rylene fragments. In another embodiment of the composite organic compound, the rylene fragments are selected from structures 1 to 21 as given in Table 1.

TABLE 1
Examples of the polycyclic organic molecule (Core) comprising rylene fragments

| | |
|---|---|
| 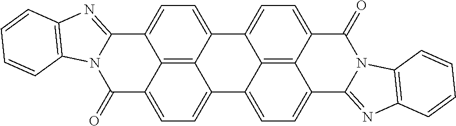 [SMILES: O=c1c2ccc3c4ccc5c6c(ccc(c7ccc(c2c37)c2nc3ccccc3n12)c46)c(=O)n1c2ccccc2nc51] | 1 |
| 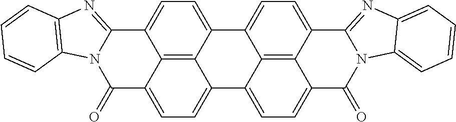 [SMILES: O=c1c2ccc3c4ccc5c(=O)n6c7ccccc7nc6c6ccc(c7ccc(c2c37)c2nc3ccccc3n12)c4c56] | 2 |
| 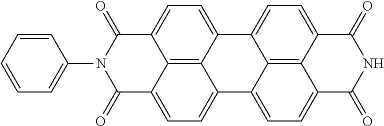 [SMILES: O=C1NC(=O)c2ccc3c4ccc5c6c(ccc(c7ccc1c2c73)c64)C(=O)N(c1ccccc1)C5=O] | 3 |
| 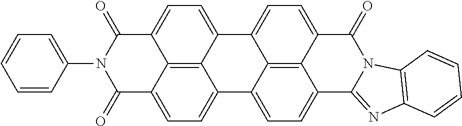 [SMILES: O=C1c2ccc3c4ccc5c(=O)n6c7ccccc7nc6c6ccc(c7ccc(c2c37)C(=O)N1c1ccccc1)c4c56] | 4 |
| 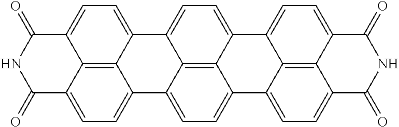 [SMILES: O=C1NC(=O)c2ccc3c4ccc5c6ccc7c8c(ccc(c9ccc(c%10ccc1c2c%103)c4c95)c86)C(=O)NC7=O] | 5 |
| 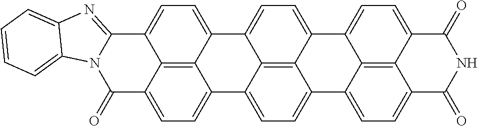 [SMILES: O=C1NC(=O)c2ccc3c4ccc5c6ccc7c8c(ccc(c9ccc(c%10ccc1c2c%103)c4c95)c68)c(=O)n1c2ccccc2nc71] | 6 |
| 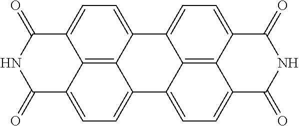 [SMILES: O=C1NC(=O)c2ccc3c4ccc5c6c(ccc(c7ccc1c2c73)c64)C(=O)NC5=O] | 7 |
| 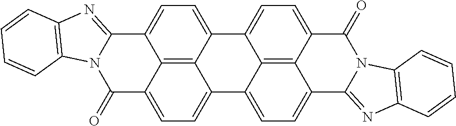 [SMILES: O=c1c2ccc3c4ccc5c6c(ccc(c7ccc(c2c37)c2nc3ccccc3n12)c46)c(=O)n1c2ccccc2nc51] | 8 |
| 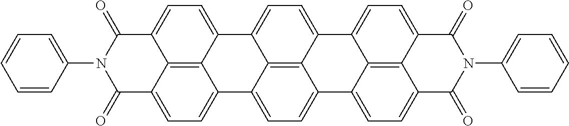 [SMILES: O=C1c2ccc3c4ccc5c6ccc7c8c(ccc(c9ccc(c%10ccc(c2c3%10)C(=O)N1c1ccccc1)c4c59)c86)C(=O)N(c1ccccc1)C7=O] | 9 |
| 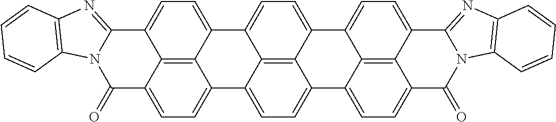 [SMILES: O=c1c2ccc3c4ccc5c6ccc7c(=O)n8c9ccccc9nc8c8ccc(c9ccc(c%10ccc(c2c3%10)c2nc3ccccc3n12)c4c59)c6c78] | 10 |
| 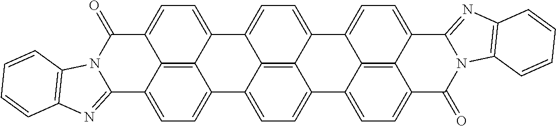 [SMILES: O=c1c2ccc3c4ccc5c6ccc7c8c(ccc(c9ccc(c%10ccc(c2c3%10)c2nc3ccccc3n12)c4c95)c68)c(=O)n1c2ccccc2nc71] | 11 |
| 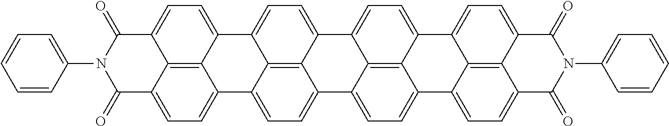 [SMILES: O=C1c2ccc3c4ccc5c6ccc7c8ccc9c%10c(ccc(c%11ccc(c%12ccc(c%13ccc(c2c3%13)C(=O)N1c1ccccc1)c4c5%12)c6c7%11)c%108)C(=O)N(c1ccccc1)C9=O] | 12 |
| 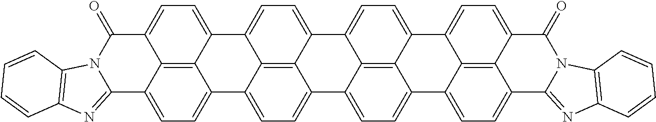 [SMILES: O=c1c2ccc3c4ccc5c6ccc7c8ccc9c(=O)n%10c%11ccccc%11nc%10c%10ccc(c%11ccc(c%12ccc(c%13ccc(c2c3%13)c2nc3ccccc3n12)c4c5%12)c6c7%11)c8c9%10] | 13 |
| 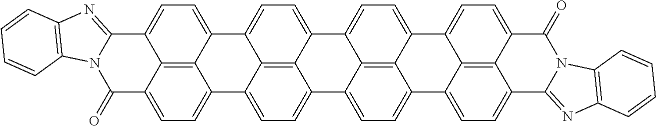 [SMILES: O=c1c2ccc3c4ccc5c6ccc7c8ccc9c%10c(ccc(c%11ccc(c%12ccc(c%13ccc(c2c3%13)c2nc3ccccc3n12)c4c5%12)c6c%117)c8%10)c(=O)n1c2ccccc2nc91] | 14 |
| 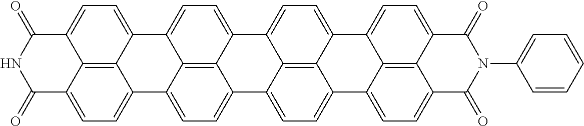 [SMILES: O=C1NC(=O)c2ccc3c4ccc5c6ccc7c8ccc9c%10c(ccc(c%11ccc(c%12ccc(c%13ccc1c2c%133)c4c%125)c6c%117)c%108)C(=O)N(c1ccccc1)C9=O] | 15 |
| 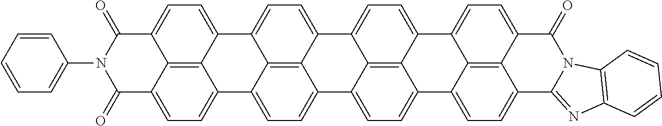 [SMILES: O=C1c2ccc3c4ccc5c6ccc7c8ccc9c(=O)n%10c%11ccccc%11nc%10c%10ccc(c%11ccc(c%12ccc(c%13ccc(c2c3%13)C(=O)N1c1ccccc1)c4c5%12)c6c7%11)c8c9%10] | 16 |
| 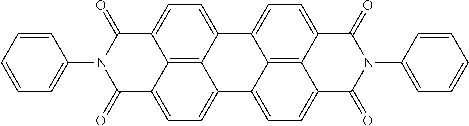 [SMILES: O=C1c2ccc3c4ccc5c6c(ccc(c7ccc(c2c37)C(=O)N1c1ccccc1)c64)C(=O)N(c1ccccc1)C5=O] | 17 |
| 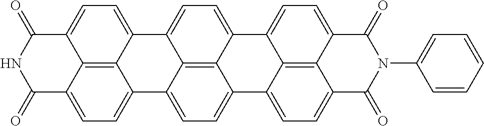 [SMILES: O=C1NC(=O)c2ccc3c4ccc5c6ccc7c8c(ccc(c9ccc(c%10ccc1c2c%103)c4c95)c86)C(=O)N(c1ccccc1)C7=O] | 18 |
| 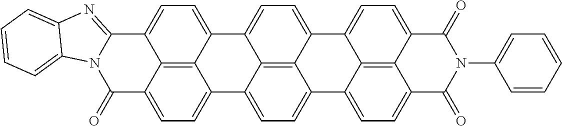 [SMILES: O=C1c2ccc3c4ccc5c6ccc7c(=O)n8c9ccccc9nc8c8ccc(c9ccc(c%10ccc(c2c3%10)C(=O)N1c1ccccc1)c4c59)c6c78] | 19 |
| 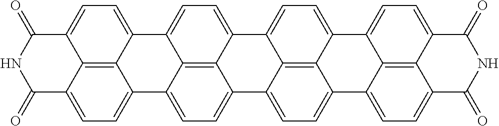 [SMILES: O=C1NC(=O)c2ccc3c4ccc5c6ccc7c8ccc9c%10c(ccc(c%11ccc(c%12ccc(c%13ccc1c2c%133)c4c%125)c6c%117)c%108)C(=O)NC9=O] | 20 |
| 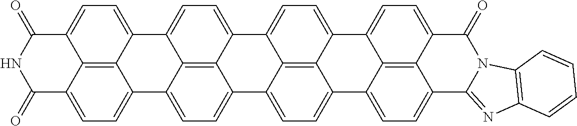 [SMILES: O=C1NC(=O)c2ccc3c4ccc5c6ccc7c8ccc9c%10c(ccc(c%11ccc(c%12ccc(c%13ccc1c2c%133)c4c%125)c6c%117)c8%10)c(=O)n1c2ccccc2nc91] | 21 |

In other implementations, the aromatic polycyclic conjugated molecule comprises an electro-conductive oligomer, such as a phenylene, thiophene, or polyacene quinine radical oligomer or combinations of two or more of these. In yet another embodiment of the composite organic compound, the electro-conductive oligomer is selected from structures 22 to 30 as given in Table 2, wherein I=2, 3, 4, 5, 6, 7, 8, 9, 10, 11 or 12, Z is ═O, ═S or ═NR5, and R5 is selected from the group consisting of unsubstituted or substituted C₁-C₁₈alkyl, unsubstituted or substituted C₂-C₁₈alkenyl, unsubstituted or substituted C₂-C₁₈alkynyl, and unsubstituted or substituted C₄-C₁₈aryl:

TABLE 2
Examples of the polycyclic organic molecule (Core)
comprising electro-conductive oligomer

| | |
|---|---|
| 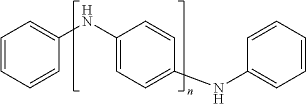 | 22 |
| 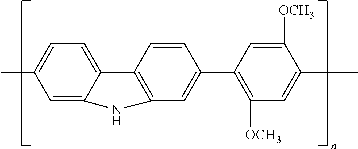 | 23 |
| 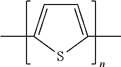 | 24 |
| 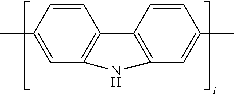 | 25 |
| 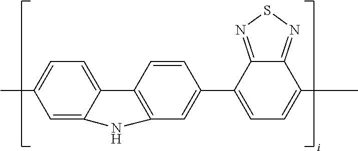 | 26 |
| 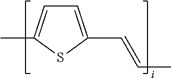 | 27 |
| 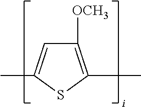 | 28 |
| 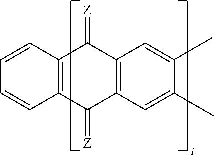 | 29 |
| 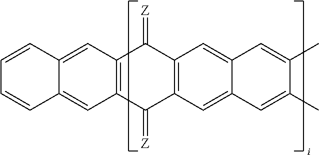 | 30 |

In some implementations, the substitute providing solubility (R1) of the composite organic compound is C_{X}Q_{2X+1}, where i≥1 and Q is hydrogen (H), fluorine (F), or chlorine (Cl). In still another embodiment of the composite organic compound, the substitute providing solubility (R1) of the composite organic compound is independently selected from alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, butyl, iso-butyl and tent-butyl groups, and the aryl group is selected from phenyl, benzyl and naphthyl groups or siloxane, and/or polyethyleneglycol as linear or branched chains.

In one embodiment of the composite organic compound, the solvent is selected from benzene, toluene, xylenes, acetone, acetic acid, methylethylketone, hydrocarbons, chloroform, carbontetrachloride, methylenechloride, dichlorethane, chlorobenzene, alcohols, nitromethan, acetonitrile, dimethylforamide, 1,4-dioxane, tetrahydrofuran (THF), methylcyclohexane (MCH), and any combination thereof.

In some embodiments, at least one electrically resistive substitute (R2) of the composite organic compound is C_{X}Q_{2X+1}, where i≥1 and Q is hydrogen (H), fluorine (F), or chlorine (Cl). In another embodiment of the composite organic compound, at least one electrically resistive substitute (R2) is selected from the list comprising —(CH₂)ₙ—CH₃, —CH((CH₂)ₙCH₃)₂) (where n≥1), alkyl, aryl, substituted alkyl, substituted aryl, branched alkyl, branched aryl, and any combination thereof and wherein the alkyl group is selected from methyl, ethyl, propyl, butyl, iso-butyl and tent-butyl groups, and the aryl group is selected from phenyl, benzyl and naphthyl groups. In yet another embodiment of the composite organic compound.

In some embodiments, at least one electrically resistive substitute (R2) is selected from the group of alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, n-butyl, iso-butyl and tent-butyl groups, and the aryl group is selected from phenyl, benzyl and naphthyl groups or siloxane, and/or polyethyleneglycol as linear or branched chains.

In some embodiments, the substitute R1 and/or R2 is connected to the aromatic polycyclic conjugated molecule (Core) via at least one connecting group. The at least one connecting group may be selected from the list comprising the following structures: 31-41 as given in Table 3, where W is hydrogen (H) or an alkyl group.

TABLE 3
Examples of the connecting group

| | |
|---|---|
| 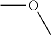 | 31 |
| 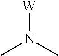 | 32 |
| 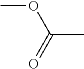 | 33 |
| 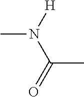 | 34 |
| 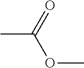 | 35 |
| 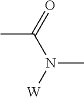 | 36 |
| 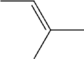 | 37 |
| 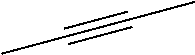 | 38 |
| 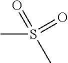 | 39 |
| 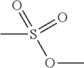 | 40 |
| 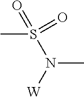 | 41 |

In some embodiments, the substitute R3 and/or R4 may be connected to the aromatic polycyclic conjugated molecule (Core) via at least one connecting group. The at least one connecting group may be selected from the list comprising CH₂, CF₂, SiR₂O, CH₂CH₂O, wherein R is selected from the list comprising H, alkyl, and fluorine. In another embodiment of the composite organic compound, the one or more ionic groups include at least one ionic group selected from the list comprising [NR₄]⁺, [PR₄]⁺ as cation and [—CO₂]⁻, [—SO₃]⁻, [—SR₅]⁻, [—PO₃R]⁻, [—PR₅]⁻ as anion, wherein R is selected from the list comprising H, alkyl, and fluorine.

Sharp polymers have hyperelectronic or ionic type polarizability. “Hyperelectronic polarization may be considered due to the pliant interaction of charge pairs of excitons, localized temporarily on long, highly polarizable molecules, with an external electric field (Roger D. Hartman and Herbert A. Pohl, “Hyper-electronic Polarization in Macromolecular Solids”, Journal of Polymer Science: Part A-1 Vol. 6, pp. 1135-1152 (1968)).” Ionic type polarization can be achieved by limited mobility of ionic parts of the tethered/partially immobilized ionic liquid or zwitterion (Q). Additionally, other mechanisms of polarization such as dipole polarization and monomers and polymers possessing metal conductivity may be used independently or in combination with hyper-electronic and ionic polarization in aspects of the present disclosure.

In some implementations, the metadielectric may include one or more Sharp polymers in the form of a composite organic compound characterized by polarizability and resistivity having the above general structural formula.

Further, characteristics of metadielectrics include a relative permittivity greater than or equal to 1,000 and resistivity greater than or equal to 10¹⁶ ohm/cm. Individually, the Sharp Polymers in a metadielectric may form column like supramolecular structures by pi-pi interaction. Said supramolecules of Sharp polymers allow formation of crystal structures of the metadielectric material. By way of using Sharp polymers in a dielectric material, polarization units are incorporated to provide the molecular material with high dielectric permeability. There are several mechanisms of polarization such as dipole polarization, ionic polarization, and hyper-electronic polarization of molecules, monomers and polymers possessing metal conductivity. All polarization units with the listed types of polarization may be used in aspects of the present disclosure. Further, Sharp polymers are composite materials which incorporate an envelope of insulating substituent groups that electrically isolate the supramolecules from each other in the dielectric crystal layer and provide high breakdown voltage of the energy storage molecular material. Said insulating substituent groups are resistive alkyl or fluro-alkyl chains covalently bonded to a polarizable core, forming the resistive envelope.

In order that the invention may be more readily understood, reference is made to the following examples, which are intended to be illustrative of the invention, but are not intended to be limiting the scope.

### EXAMPLE 2

This Example describes synthesis of one type of Sharp polymer according following structural scheme:

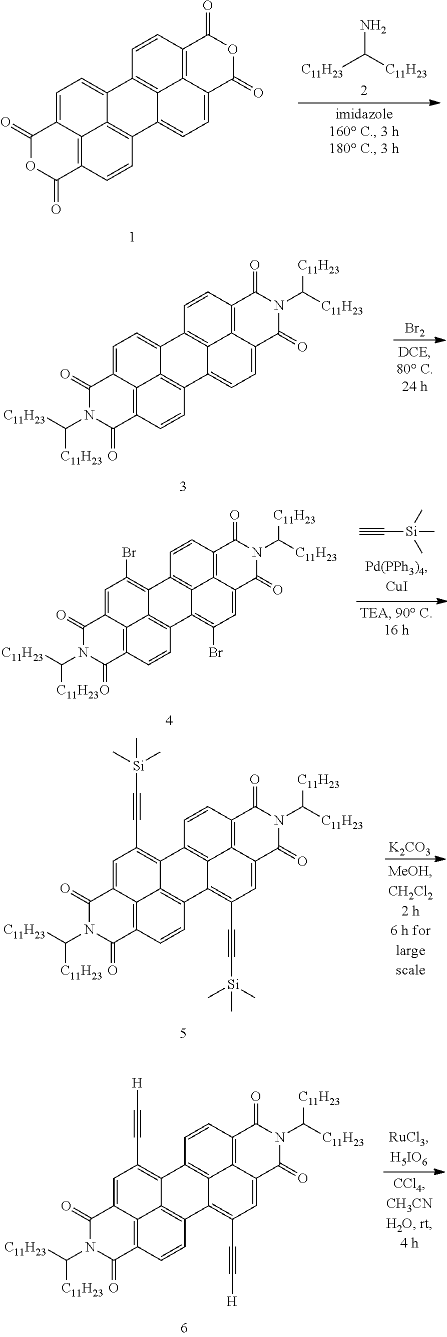

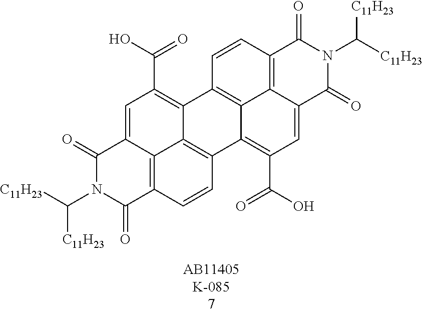

The process involved in the synthesis in this example may be understood in terms of the following five steps.

a) First step:

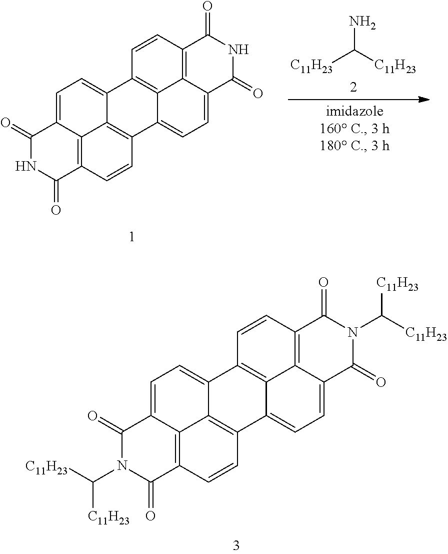

Anhydride 1 (60.0 g, 0.15 mol, 1.0 eq), amine 2 (114.4 g, 0.34 mol, 2.2 eq) and imidazole (686.0 g, 10.2 mol, 30 eq to 2) were mixed well into a 500 mL of round-bottom flask equipped with a bump-guarder. The mixture was degassed three times, stirred at 160° C. for 3 hr, 180° C. for 3hr, and cooled to rt. The reaction mixture was crushed into water (1000 mL) with stirring. Precipitate was collected with filtration, washed with water (2×500 mL), methanol (2×300 mL) and dried on high vacuum. The crude product was purified by flash chromatography column (CH₂Cl₂/hexane=1/1) to give 77.2 g (48.7%) of the desired product 3 as an orange solid. ¹H NMR (300 MHz, CDCl₃) δ 8.65-8.59 (m, 8H), 5.20-5.16 (m, 2H), 2.29-2.22 (m, 4H), 1.88-1.82 (m, 4H), 1.40-1.13 (m, 64H), 0.88-0.81 (t, 12H). Rf=0.68 (CH₂Cl₂/hexane=1/1).

b) Second step:

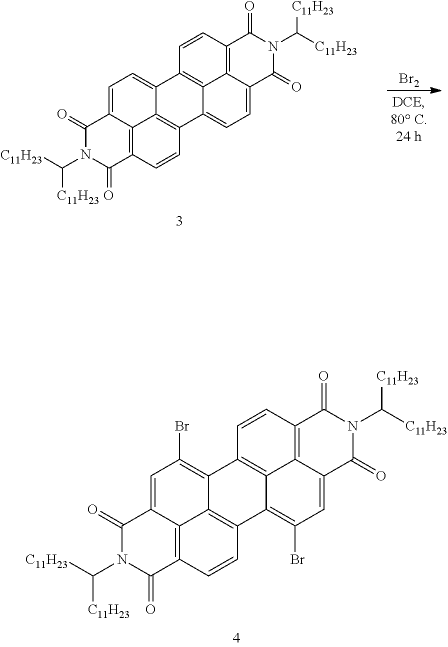

To a solution of the diimide 3 (30.0 g, 29.0 mmol, 1.0 eq) in dichloroethane (1500 mL) was added bromine (312.0 g, 1.95 mol, 67.3 eq). The resulting mixture was stirred at 80° C. for 36 hr, cooled, washed with 10% NaOH (aq, 2×1000 mL), water (100 ml), dried over Na₂SO₄, filtered and concentrated. The crude product was purified by flash chromatography column (CH₂Cl₂/hexanes=1/1) to give 34.0 g (98.2%) of the desired product 4 as a red solid. ¹H NMR (300 MHz, CDCl₃) δ 9.52 (d, 2H), 8.91 (bs, 2H), 8.68 (bs, 2H), 5.21-5.13 (m, 2H), 2.31-2.18 (m, 4H), 1.90-1.80 (m, 4H), 1.40-1.14 (m, 64H), 0.88-0.81 (t, 12H). Rf=0.52 (CH₂Cl₂/hexanes=1/1).

c) Third step

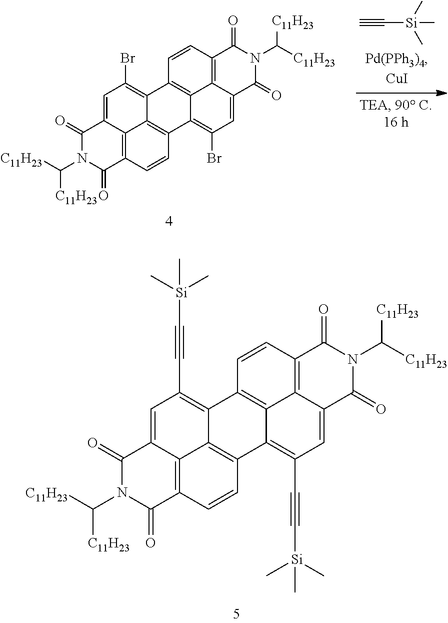

To a solution of the di-bromide 4 (2.0 g, 1.68 mmol, 1.0 eq) in triethylamine (84.0 mL) was added CuI (9.0 mg, 0.048 mmol, 2.8 mol %) and (trimethylsilyl)acetylene (80.49 g, 5.0 mmol, 3.0 eq). The mixture was degassed three times. Catalyst Pd(PPh₃)₄ (98.0 mg, 0.085 mmol, 5.0 mol %) was added. The mixture was degassed three times, stirred at 90° C. for 24 hr, cooled, passed through a pad of Celite, and concentrated. The crude product was purified by flash chromatography column (CH₂Cl₂/hexane=1/1) to give 1.8 g (87.2%) of the desired product 5 as a dark-red solid. ¹H NMR (300 MHz, CDCl₃) δ 10.24-10.19 (m, 2H), 8.81 (bs, 2H), 8.65 (bs, 2H), 5.20-5.16 (m, 2H), 2.31-2.23 (m, 4H), 1.90-1.78 (m, 4H), 1.40-1.15 (m, 72H), 0.84-0.81 (t, 12H), 0.40 (s, 18H). Rf=0.72 (CH₂Cl₂/hexane=1/1).

d) Fourth step

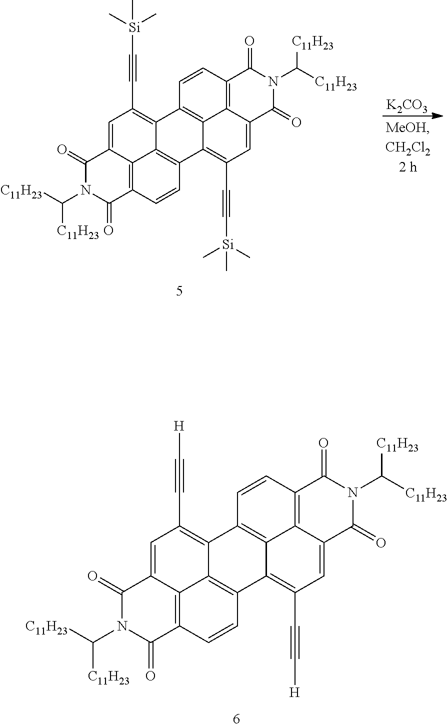

To a solution of diimide 5 (1.8 g, 1.5 mmol, 1.0 eq) in a mixture of MeOH/DCM (40.0 mL/40.0 mL) was added K₂CO₃ (0.81 g, 6.0 mmol, 4.0 eq). The mixture was stirred at room temperature for 1.5 hr, diluted with DCM (40.0 mL), washed with water, brine, dried over Na₂SO₄, filtered and concentrated. The crude product was purified by flash chromatography column (CH₂Cl₂) to give 1.4 g (86.1%) of the desired product 6 as a dark-red solid. ¹H NMR (300 MHz, CDCl₃) δ 10.04-10.00 (m, 2H), 8.88-8.78 (m, 2H), 8.72-8.60 (m, 2H), 5.19-5.14 (m, 2H), 3.82-3.80 (m, 2H), 2.31-2.23 (m, 4H), 1.90-1.78 (m, 4H), 1.40-1.05 (m, 72H), 0.85-0.41 (t, 12H). Rf=0.62 (CH₂Cl₂).

e) Fifth step

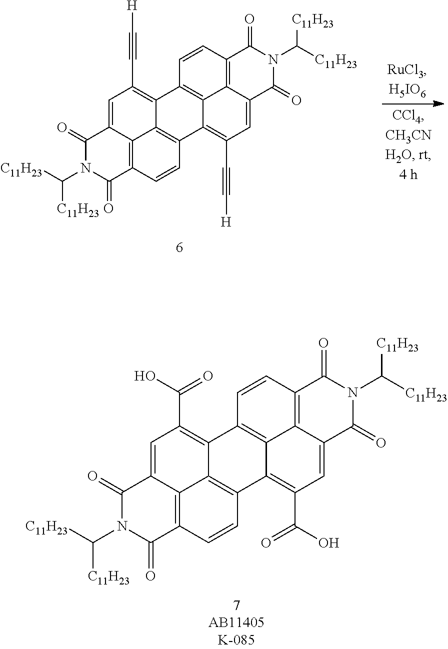

To a suspension of alkyne 6 (1.4 g, 1.3 mmol, 1.0 eq) in a mixture of CCl₄/CH₃CN/H₂O (6 mL/6 mL/12 mL) was added periodic acid (2.94 g, 12.9 mmol, 10.0 eq) and RuCl₃ (28.0 mg, 0.13 mmol, 10 mol %). The mixture was stirred at room temperature under nitrogen for 4 hours, diluted with DCM (50 mL), washed with water, brine, dried over Na₂SO₄, filtered and concentrated. The crude product was purified by flash chromatography column (10% MeOH/CH₂Cl₂) to give 1.0 g (68.5%) of the desired product 7 as a dark-red solid. ¹H NMR (300 MHz, CDCl₃) δ 8.90-8.40 (m, 6H), 5.17-5.00 (m, 2H), 2.22-2.10 (m, 4H), 1.84-1.60 (m, 4H), 1.41-0.90 (m, 72H), 0.86-0.65 (t, 12H). Rf=0.51 (10% MeOH/CH₂Cl₂).

### EXAMPLE 3

This Example describes synthesis of a Sharp polymer according following structural scheme:

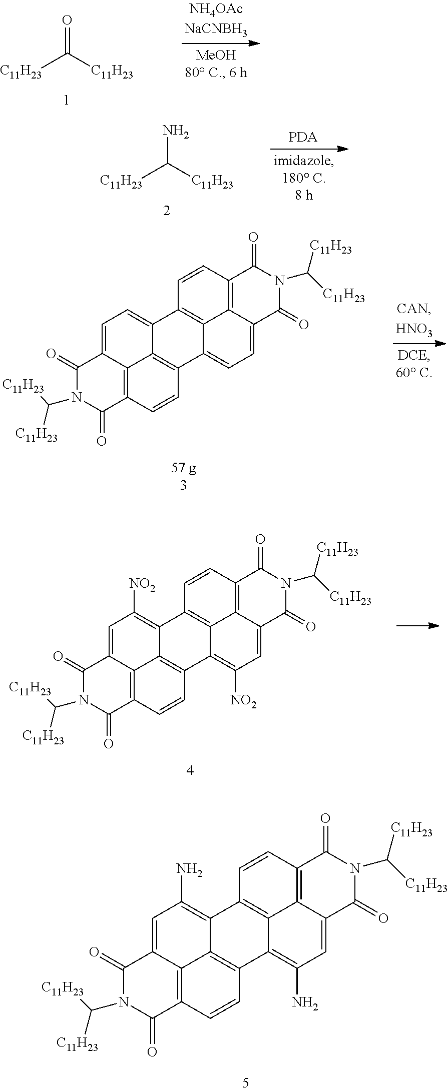

The process involved in the synthesis in this example may be understood in terms of the following four steps.

a) First sten:

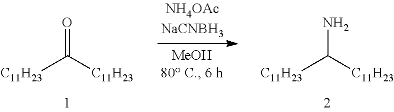

To a solution of the ketone 1 (37.0 g, 0.11 mol, 1.0 eq) in methanol (400 mL) was added ammonium acetate (85.3 g, 1.11 mol, 10.0 eq) and NaCNBH₃ (28.5 g, 0.44 mol, 4.0 eq) in portions. The mixture was stirred at reflux for 6 hours, cooled to room temperature and concentrated. Sat. NaHCO₃ (500 mL) was added to the residue and the mixture was stirred at room temperature for 1 hour. Precipitate was collected by filtration, washed with water (4×100 mL), dried on a high vacuum to give 33.6 g (87%) of the amine 2 as a white solid.

b) Second step:

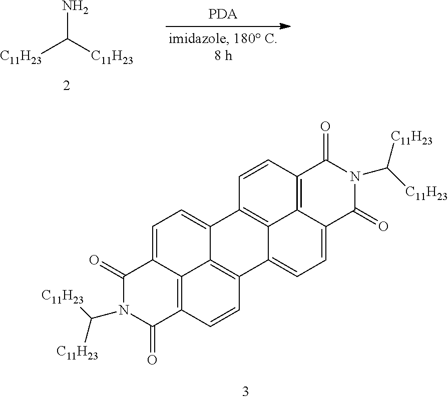

Mixed well the amine 2 (20.0 g, 58.7 mmol, 2.2 equ), 3,4,9,10-perylenetetracarboxylic dianhydride (10.5 g, 26.7 mmol, 1.0 eq) and imidazole (54.6 g, 0.80 mmol, 30 eq to diamine) into a 250 mL round-bottom flask equipped with a rotavap bump guard. The mixture was degased (vacuum and fill with N₂) three times and stirred at 160° C. for 6 hrs. After cooling to rt, the reaction mixture was crushed into water (700 mL), stirred for 1 hr, and filtered through a filter paper to collected precipitate which was washed with water (3×300 mL) and mthanol (3×300 mL), dried on a high vacum to give 23.1 g (83.5%) of the diamidine 3 as a orange solid. Pure diamidine 3 (20.6 g) was obtained by flash chromatography column (DCM/hexanes=1/1).

c) Third step:

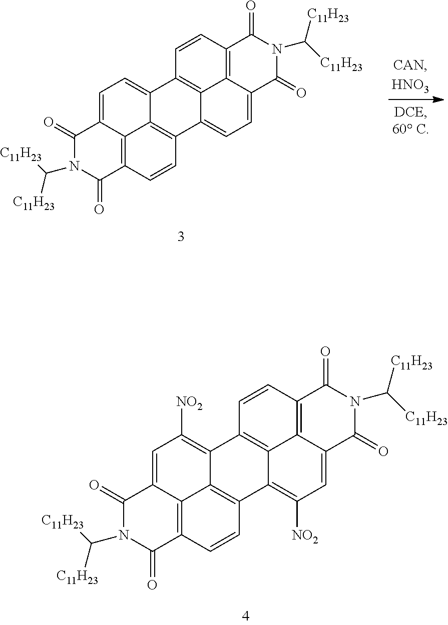

To DCE (2.0 L) was added compound 3 (52.0 g, 50.2 mmol, 1.0 eq), acetic acid (500 mL) and fuming nitric acid (351.0 g, 5.0 mol, 100.0 eq) with caution. To the mixture was added ammonium cerium(IV) nitrate (137.0 g, 0.25 mol, 5.0 eq). The reaction was stirred at 60° C. for 48 hrs. After cooling to rt, the reaction mixture was crushed into water (1.0 L). The organic phase was washed with water (2×1.0 L), saturated NaHCO3 solution (1×1.0 L) and brine (1×1.0 L), dried over sodium sulfate, filtered and concentrated. The residue was purified with column chromatography to give 46.7g (82%) of compound 4 as a dark red solid. ¹H NMR (300 MHz, CDCl₃) 6 0.84 (t, 12H), 1.26 (m, 72H), 1.83 (m, 4H), 2.21 (m, 4H), 5.19 (m, 2H), 8.30 (m, 2H), 8.60-8.89 (m, 4H).

d) Fourth step:

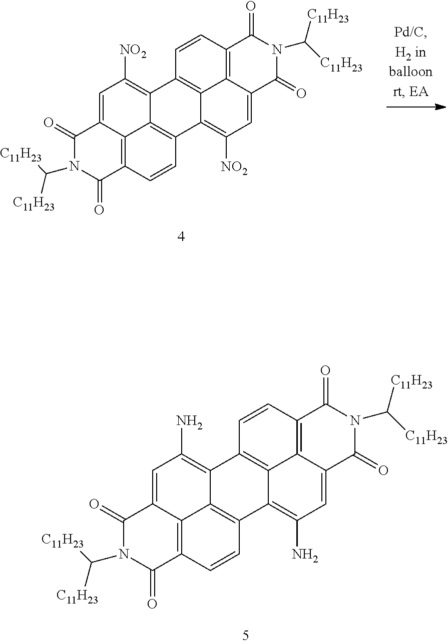

A mixture of compound 4 (25 g, 22.2 mmol, 1.0 eq) and Pd/C (2.5 g, 0.1 eq) in EtOAc (125.0 mL) was stirred at room temperature for 1 hour. The solid was filtered off (Celite) and washed with EtOAc (5 mL×2). The filtrate was concentrated to afford the compound 5 (23.3 g, 99%) as a dark blue solid. ¹H NMR (300 MHz, CDCl₃) δ 0.84 (t, 12H), 1.24 (m, 72H), 1.85 (m, 4H), 2.30 (m, 4H), 5.00 (s, 2H), 5.10 (s, 2H), 5.20 (m, 2H), 7.91-8.19 (dd, 2H), 8.40-8.69 (dd, 2H), 8.77-8.91 (dd, 2H).

Furuta co-polymers and para-Furuta polymers (herein referred to collectively as Furuta Polymers unless otherwise specified) are polymeric compounds with insulating tails, and linked/tethered/partially immobilized polarizable ionic groups. The insulating tails are hydrocarbon (saturated and/or unsaturated), fluorocarbon, siloxane, and/or polyethylene glycol linear or branched chains covalently bonded to the co-polymer backbone. The tails act to insulate the polarizable tethered/partially immobilized ionic molecular components and ionic pairs from other ionic groups and ionic group pairs on the same or parallel co-polymers, which favorably allows discrete polarization of counter ionic liquid pairs or counter Q groups (i.e. polarization of cationic liquid and anionic liquid tethered/partially immobilized to parallel Furuta polymers) with limited or no interaction of ionic fields or polarization moments of other counter ionic group pairs partially immobilized on the same or parallel co-polymer chains. Further, the insulating tails electrically insulate supra-structures of Furuta polymers from each other. Parallel Furuta polymers may arrange or be arranged such that counter ionic groups (i.e. tethered/partially immobilized ionic groups (Qs) of cation and anion types (sometimes known as cationic Furuta polymers and anionic Furuta polymers)) are aligned opposite from one another. In some implementations, the metadielectric layer may include two or more Furuta polymers, including a Furuta polymer having an immobilized ion liquid group of a cationic or anionic type.

A Furuta co-polymer has the following general structural formula:

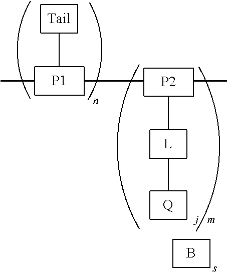

wherein backbone structure of the co-polymer comprises structural units of first type P1 and structural units of second type P2 both of which randomly repeat and are independently selected from the list comprising acrylic acid, methacrylate, repeat units of polypropylene (—[CH₂—CH(CH₃)]—), repeat units of polyethylene (—[CH₂]—), siloxane, or repeat units of polyethylene terephthalate (sometimes written poly(ethylene terephthalate)) for which the repeat unit may be expressed as —CH₂—CH₂—O—CO—C₆H₄—CO—O—. Parameter n is the number of the P1 structural units in the backbone structure which is in the range from 3 to 100,000 and m is number of the P2 structural units in the backbone structure which is in the range from 3 to 100,000. Further, the first type structural unit (P1) has a resistive substitute Tail which is oligomers of polymeric material with HOMO-LUMO gap no less than 2 eV. Additionally, the second type of structural units (P2) has an ionic functional group Q which is connected to P2 via a linker group L. The parameter j is a number of functional groups Q attached to the linker group L, which may range from 0 to 5. Wherein the ionic functional group Q comprises one or more ionic liquid ions (from the class of ionic compounds that are used in ionic liquids), zwitterions, or polymeric acids. Further, an energy interaction of the ionic Q groups may be less than kT, where k is Boltzmann constant and T is the temperature of environment. Still further, parameter B is a counter ion which is a molecule or molecules or oligomers that can supply the opposite charge to balance the charge of the co-polymer. Wherein, s is the number of the counter ions.

The present disclosure provides an organic co-polymeric compound having the structure described above. In one embodiment of the organic co-polymeric compound, the resistive substitute Tails are independently selected from the list comprising oligomers of polypropylene (PP), oligomers of polyethylene terephthalate (PET), oligomers of polyphenylene sulfide (PPS), oligomers of polyethylene naphthalate (PEN), oligomers of polycarbonate (PP), polystyrene (PS), and oligomers of polytetrafluoroethylene (PTFE). In another embodiment of the organic co-polymeric compound, the resistive substitutes Tail are independently selected from alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, butyl, iso- butyl and tent-butyl groups, and the aryl group is selected from phenyl, benzyl and naphthyl groups. The resistive substitute Tail may be added after polymerization.

In yet another aspect of the present disclosure, it is preferable that the HOMO-LUMO gap is no less than 4 eV. In still another aspect of the present disclosure, it is even more preferable that the HOMO-LUMO gap is no less than 5 eV. The ionic functional group Q comprises one or more ionic liquid ions from the class of ionic compounds that are used in ionic liquids, zwitterions, or polymeric acids. The energy of interaction between Q group ions on discrete P₂ structural units may be less than kT, where k is Boltzmann constant and T is the temperature of environment. The temperature of environment may be in range between −60 □ of and 150 □. The preferable range of temperatures is between −40 □ and 100 □. Energy interaction of the ions depends on the effective radius of ions. Therefore, by increasing the steric hindrance between ions it is possible to reduce energy of interaction of ions. In one embodiment of the present invention, at least one ionic liquid ion is selected from the list comprising [NR₄]⁺, [PR₄]⁺ as cation and [—CO₂]⁻, [—SR₃]⁻, [—PO₃R]⁻, [—PR₅]⁻as anion, wherein R is selected from the list comprising H, alkyl, and fluorine. The functional group Q may be charged after or before polymerization. In another embodiment of the present invention, the linker group L is oligomer selected from structures 42 to 47 as given in Table 3.

TABLE 3
Examples of the oligomer linker group

| | |
|---|---|
| 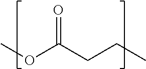 | 42 |
| 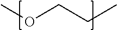 | 43 |
| 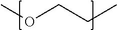 | 44 |
| 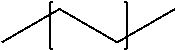 | 45 |
| 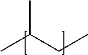 | 46 |
| 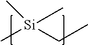 | 47 |

In yet another embodiment of the present invention, the linker group L is selected from structures 48 to 57 as given in Table 4.

TABLE 4
Examples of the linker group

| | |
|---|---|
| 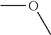 | 48 |
| 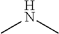 | 49 |
| 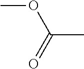 | 50 |
| 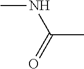 | 51 |
| 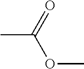 | 52 |
| 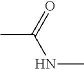 | 53 |
| 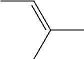 | 54 |
| 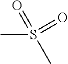 | 55 |
| 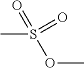 | 56 |
| 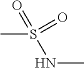 | 57 |

In yet another embodiment of the present invention, the linker group L may be selected from the list comprising CH₂, CF₂, SiR₂O, and CH2CH2O, wherein R is selected from the list comprising H, alkyl, and fluorine. The ionic functional group Q and the linker groups L may be added after polymerization.

In another aspect, the present disclosure provides a dielectric material (sometimes called a metadielectric) comprising of one or more of the class of Furuta polymers comprising protected or hindered ions of zwitterion, cation, anion, or polymeric acid types described hereinabove. The metadielectric material may be a mixture of zwitterion type Furuta polymers, or positively charged (cation) Furuta polymers and negatively charged (anion) Furuta polymers, polymeric acid Furuta polymers, or any combination thereof. The mixture of Furuta polymers may form or be induced to form supra-structures via hydrophobic and ionic interactions. By way of example, but not limiting in scope, the cation on a positively charged Furuta polymer replaces the B counter ions of the anion on a negatively charged Furuta polymer parallel to the positively charged Furuta polymer and vice versa; and the resistive Tails of neighboring Furuta polymers further encourages stacking via van der Waals forces, which increases ionic group isolation. Metadielectrics comprising both cationic and anionic Furuta polymers have a 1:1 ratio of cationic and anionic Furuta polymers.

The Tails of hydrocarbon (saturated and/or unsaturated), fluorocarbon, siloxane, and/or polyethylene glycol linear or branched act to insulate linked/tethered/partially immobilized polarizable ionic liquids, zwitterions, or polymeric acids (ionic Q groups). The Tails insulate the ionic Q groups from other ionic Q groups on the same or parallel Furuta polymer via steric hindrance of the ionic Q groups' energy of interaction, which favorably allows discrete polarization of the ionic Q groups (i.e. polarization of cationic liquid and anionic liquid tethered/partially immobilized to parallel Furuta polymers). Further, the Tails insulate the ionic groups of supra-structures from each other. Parallel Furuta polymers may arrange or be arranged such that counter ionic liquids (i.e. tethered/partially immobilized ionic liquids (Qs) of cation and anion types) are aligned opposite from one another (sometimes known as cationic Furuta polymers and anionic Furuta polymers).

The Furuta polymers have hyperelectronic or ionic type polarizability. “Hyperelectronic polarization may be considered due to the pliant interaction of charge pairs of excitons, localized temporarily on long, highly polarizable molecules, with an external electric field (Roger D. Hartman and Herbert A. Pohl, “Hyper-electronic Polarization in Macromolecular Solids”, Journal of Polymer Science: Part A-1 Vol. 6, pp. 1135-1152 (1968)).” Ionic type polarization can be achieved by limited mobility of ionic parts of the tethered/partially immobilized ionic liquid or zwitterion (Q). Additionally, other mechanisms of polarization such as dipole polarization and monomers and polymers possessing metal conductivity may be used independently or in combination with hyper-electronic and ionic polarization in aspects of the present disclosure.

Further, a metadielectric layer may be comprised of one or more types of zwitterion Furuta polymer and/or selected from the anionic Q⁺ group types and cationic Q⁻ group types and/or polymeric acids, having the general configuration of Furuta polymers:

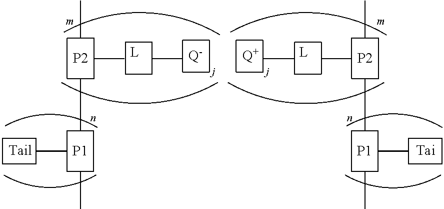

In order that the invention may be more readily understood, reference is made to the following examples of synthesis of Furuta co-polymers, which are intended to be illustrative of the invention, but are not intended to be limiting the scope.

### EXAMPLE 4

Carboxylic acid co-polymer P002. To a solution of 1.02 g (11.81 mmol) of methacrylic acid and 4.00 g (11.81 mmol) of stearylmethacrylate in 2.0 g isopropanol was added a solution of 0.030 g 2,2′-azobis(2-methylpropionitrile) (AIBN) in 5.0 g of toluene. The resulting solution was heated to 80 □ for 20 hours in a sealed vial, after which it became noticeably viscous. NMR shows <2% remaining monomer. The solution was used without further purification in film formulations and other mixtures.

### EXAMPLE 5

Amine co-polymer P011. To a solution of 2.52 g (11.79 mmol) of 2-(diisopropylamino)ethyl methacrylate and 3.00 g (11.79 mmol) of laurylmethacrylate in 2.0 g toluene was added a solution of 0.030 g 2,2′-azobis(2-methylpropionitrile) (AIBN) in 4.0 g of toluene. The resulting solution was heated to 80 □ for 20 hours in a sealed vial, after which it became noticeably viscous. NMR shows <2% remaining monomer. The solution was used without further purification in film formulations and other mixtures.

### EXAMPLE 6

Carboxylic acid co-polymer and amine co-polymer mixture. 1.50 g of a 42 wt % by solids solution of P002 was added to 1.24 g of a 56 wt % solution of P011 with lg of isopropanol and mixed at 40 □ for 30 minutes. The solution was used without further purification.

A para-Furuta polymer has repeat units of the following general structural formula:

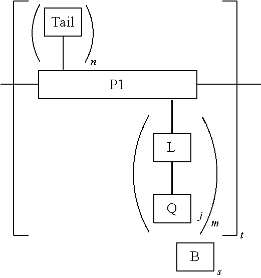

wherein a structural unit P comprises a backbone of the copolymer, which is independently selected from the list comprising acrylic acid, methacrylate, repeat units for polypropylene (PP) (—[CH₂—CH(CH₃)]—), repeat units for polyethylene (PE) (—[CH₂]—), siloxane, or repeat units of polyethylene terephthalate (sometimes written poly(ethylene terephthalate)) for which the repeat unit may be expressed as —CH₂—CH₂—O—CO—C₆H₄—CO—O—. Wherein the first type of repeat unit (Tail) is a resistive substitute in the form of an oligomer of a polymeric material. The resistive substitute preferably has a HOMO-LUMO gap no less than 2 eV. The parameter n is a number of Tail repeat units on the backbone P structural unit, and is in the range from 3 to 100,000. Further, the second type of repeat units (-L-Q) include an ionic functional group Q which is connected to the structural backbone unit (P) via a linker group L, and m is number of the -L-Q repeat units in the backbone structure which is in the range from 3 to 100,000. Additionally, the ionic functional group Q comprises one or more ionic liquid ions (from the class of ionic compounds that are used in ionic liquids), zwitterions, or polymeric acids. An energy of interaction of the ionic Q groups may be less than kT, where k is Boltzmann constant and T is the temperature of environment. Still further, the parameter t is average of para-Furuta polymer repeat units, ranging from 6 to 200,000. Wherein B's are counter ions which are molecules or oligomers that can supply the opposite charge to balance the charge of the co-polymer, s is the number of the counter ions.

In some implementations, the resistive substitute Tails are independently selected from the list comprising polypropylene (PP), polyethylene terephthalate (PET), polyphenylene sulfide (PPS), polyethylene naphthalate (PEN), polycarbonate (PP), polystyrene (PS), and polytetrafluoroethylene (PTFE). In another embodiment of the organic polymeric compound, the resistive substitutes Tail are independently selected from alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, butyl, iso- butyl and tert-butyl groups, and the aryl group is selected from phenyl, benzyl and naphthyl groups. The resistive substitute Tail may be added after polymerization. In yet another embodiment of the present disclosure, it is preferable that the HOMO-LUMO gap is no less than 4 eV. In still another embodiment of the present disclosure, it is even more preferable that the HOMO-LUMO gap is no less than 5 eV. The ionic functional group Q comprises one or more ionic liquid ions from the class of ionic compounds that are used in ionic liquids, zwitterions, or polymeric acids. Energy of interaction between Q group ions on discrete P structural units may be less than kT, where k is Boltzmann constant and T is the temperature of environment. The temperature of environment may be in range between −60 □ and 150 □. The preferable range of temperatures is between −40 □ and 100 □. Energy interaction of the ions depends on the effective radius of ions. Therefore, by increasing the steric hindrance between ions it is possible to reduce energy of interaction of ions. In one embodiment of the present invention, at least one ionic liquid ion is selected from the list comprising [NR₄]⁺, [PR₄]⁺ as cation and [—CO₂]⁺, [—SR₃]⁺, [—PO₃R]⁺, [—PR₅]⁺ as anion, wherein R is selected from the list comprising H, alkyl, and fluorine. The functional group Q may be charged after or before polymerization. In another embodiment of the present invention, the linker group L is oligomer selected from structures 42 to 47 as given in Table 3 or structures 48 to 57 in Table 4.

In some implementations, the linker group L is selected from the list comprising CH₂, CF₂, SiR₂O, and CH₂CH₂O, wherein R is selected from the list comprising H, alkyl, and fluorine. The ionic functional group Q and the linker groups L may be added after polymerization.

In some implementations, the metadielectric includes one or more of the class of para-Furuta polymers comprising protected or hindered ions of zwitterion, cationic liquid ions, anionic liquid ions, or polymeric acid types described hereinabove. The metadielectric material may be a mixture of zwitterion type para-Furuta polymers, or positively charged (cation) para-Furuta polymers and negatively charged (anion) para-Furuta polymers, polymeric acid para-Furuta polymers, or any combination thereof. The mixture of para-Furuta polymers may form or be induced to form supra-structures via hydrophobic and ionic interactions. By way of example, but not limiting in scope, the cation(s) on a positively charged para-Furuta polymer replaces the B counter ions of the anion(s) on a negatively charged para-Furuta polymer parallel to the positively charged para-Furuta polymer and vice versa; and the resistive Tails of neighboring para-Furuta polymers further encourages stacking via van der Waals forces, which increases ionic group isolation. Metadielectrics comprising both cationic and anionic para-Furuta polymers preferably have a 1:1 ratio of cationic and anionic para-Furuta polymers.

The Tails of hydrocarbon (saturated and/or unsaturated), fluorocarbon, siloxane, and/or polyethylene glycol linear or branched act to insulate linked/tethered/partially immobilized polarizable ionic liquids, zwitterions, or polymeric acids (ionic Q groups). The Tails insulate the ionic Q groups from other ionic Q groups on the same or parallel para-Furuta polymer via steric hindrance of the ionic Q groups' energy of interaction, which favorably allows discrete polarization of the ionic Q groups (i.e. polarization of cationic liquid and anionic liquid tethered/partially immobilized to parallel para-Furuta polymers). Further, the Tails insulate the ionic groups of supra-structures from each other. Parallel para-Furuta polymers may arrange or be arranged such that counter ionic liquids (i.e. tethered/partially immobilized ionic liquids (Qs) of cation and anion types) are aligned opposite from one another (sometimes known as cationic para-Furuta polymers and anionic para-Furuta polymers).

The para-Furuta polymers have hyperelectronic or ionic type polarizability. “Hyperelectronic polarization may be considered due to the pliant interaction of charge pairs of excitons, localized temporarily on long, highly polarizable molecules, with an external electric field [.] (Roger D. Hartman and Herbert A. Pohl, “Hyper-electronic Polarization in Macromolecular Solids”, Journal of Polymer Science: Part A-1 Vol. 6, pp. 1135-1152 (1968)).” Ionic type polarization can be achieved by limited mobility of ionic parts of the tethered/partially immobilized ionic liquid or zwitterion (Q). Additionally, other mechanisms of polarization such as dipole polarization and monomers and polymers possessing metal conductivity may be used independently or in combination with hyper-electronic and ionic polarization in aspects of the present disclosure.

Further, a metadielectric layer may be comprised of one or more types of zwitterion para-Furuta polymer and/or selected from the anionic Q group types and cationic Q group types and/or polymeric acids, which may have the following general arrangement of para-Furuta polymers:

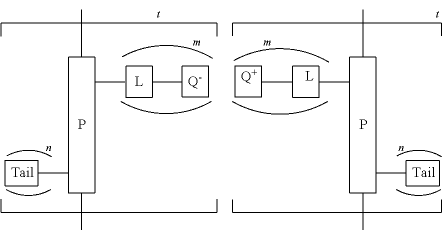

A metadielectric is defined here as a dielectric material comprised of one or more types of structured polymeric materials (SPMs) having a relative permittivity greater than or equal to 1000 and resistivity greater than or equal to 10¹³ ohm/cm. Individually, the SPMs in a metadielectric may form column like supramolecular structures by pi-pi interaction or hydrophilic and hydrophobic interactions. Said supramolecules of SPMs may permit formation of crystal structures of the metadielectric material. By way of using SPMs in a dielectric material, polarization units are incorporated to provide the molecular material with high dielectric permeability. There are several mechanisms of polarization such as dipole polarization, ionic polarization, and hyper-electronic polarization of molecules, monomers and polymers possessing metal conductivity. All polarization units with the listed types of polarization may be used in aspects of the present disclosure. Further, SPMs are composite materials which incorporate an envelope of insulating substituent groups that electrically isolate the supramolecules from each other in the dielectric layer and provide high breakdown voltage of the energy storage molecular material. Said insulating substituent groups are hydrocarbon (saturated and/or unsaturated), fluorocarbon, siloxane, and/or polyethylene glycol linear or branched chains covalently bonded to a polarizable core or co-polymer backbone, forming the resistive envelope.

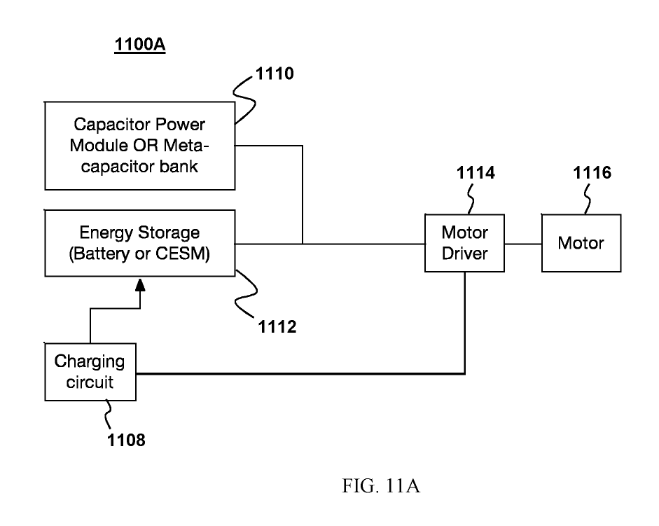

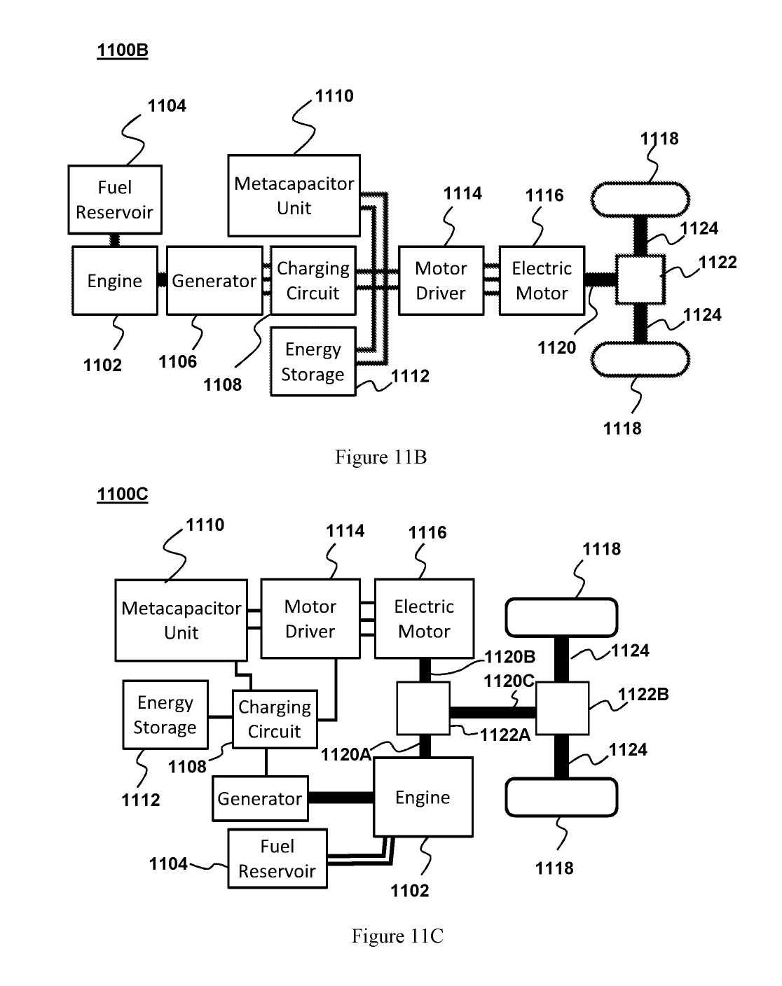

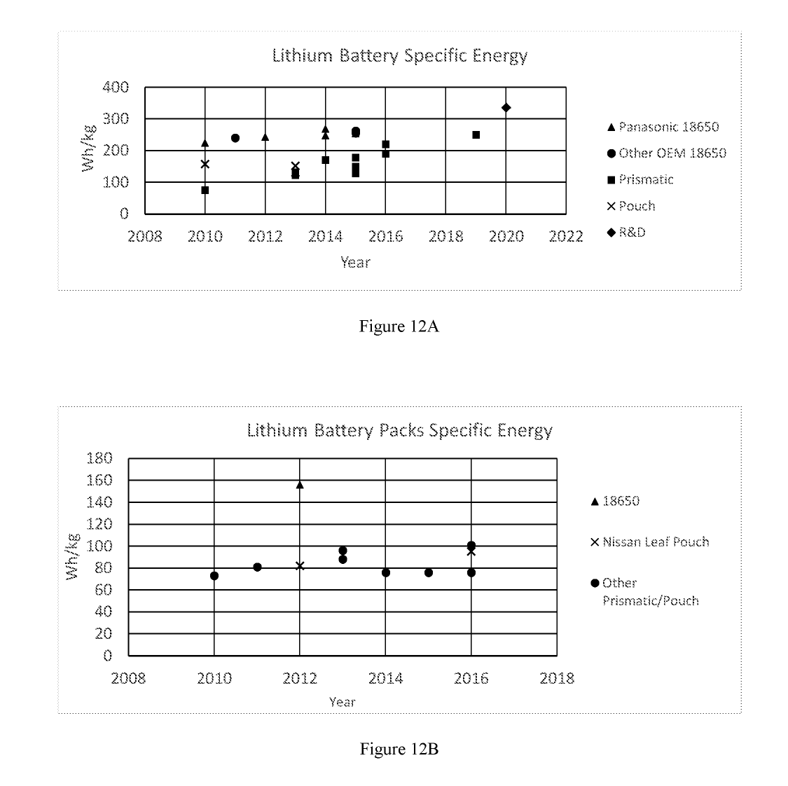

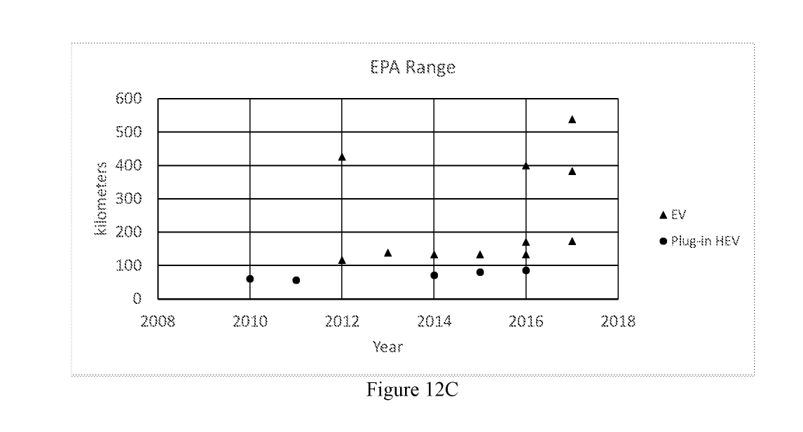

Aspects of the present disclosure pertain to both electric and hybrid vehicles. FIG. 11A schematically depicts the architecture of an electric vehicle power system 1100A that uses one or more metacapacitors for electrical energy storage according to aspects of the present disclosure. In the system 1100A, a charging circuit provides electrical energy to a power module 1110 (e.g., a capacitor power module CPM or metacapacitor bank) and an energy storage module 1112, which may be a battery or a CESM. A motor driver circuit 1114 selectively provides electrical energy from the power module 1110 or energy storage module 1112 (or possibly both) to power an electric traction motor 1116. The motor converts the electrical power supplied by the charging circuit to mechanical power that drives the vehicle, e.g., by driving the vehicle's wheels (not shown). The motor driver 1114 receives a DC input voltage and outputs either DC or AC (e.g., 3 phase AC), depending on type of motor 1116. The motor driver circuit may also be configured to function as a speed/torque controller, e.g., by outputting pulse-width modulated power signals to control the electric current (which in turn controls the motor's torque) at variable frequencies to control rotational velocity. The motor driver circuit 1114 or possibly the electric motor 1116 itself may also be electrically coupled to the charging circuit 1109 in order to charge the CPM 1110 and/or energy storage module 1112 through regenerative braking.

Aspects of the present disclosure include implementations in which the energy storage module is a battery and the capacitor power module (CPM) is a metacapacitor-based CESM. Using a lightweight metacapacitor CPM allows one to decouple the power and energy optimization of batteries. The battery chemistry may be selected based on energy density and the CPM may be selected for optimized for acceleration and regenerative braking.

In the case of hybrid vehicles they may either be series hybrids as shown in FIG. 11B or parallel hybrids as shown in FIG. 11C. In FIG. 11B and FIG. 11C the converter shown in each example connecting DC bus to the electric motor is actually a motor driver, not a DC-DC converter. This takes a DC input voltage and outputs either 3 phase AC or DC, depending on type of motor. But it is also a speed/torque controller, so it outputs pulse-width modulated power signals to control current (torque) at variable frequencies to control rotational velocity.

In the series hybrid 1100B shown in FIG. 11B, an internal combustion engine 1102 runs on fuel, e.g., gasoline or diesel, from a reservoir 1104. The engine 1102 converts chemical energy in the fuel to mechanical energy that drives an electric generator 1106 to convert kinetic energy from the engine to electrical energy. The generator 1106 is coupled to a charging circuit 1108, which is in turn connected to a metacapacitor unit 1110, an energy storage unit 1112 and a motor driver circuit 1114. The motor driver circuit 1114 provides electrical power from the metacapacitor unit 1110 and/or energy storage unit 1112 to an electric traction motor 1116, which in turn provides mechanical power to drive wheels 1118. The motor 1116 may be connected to the wheels 1118 by a drive train, such as a drive shaft 1120, differential 1122, and one or more drive axles 1124. Alternatively, the motor 1116 may be directly coupled to one or more of the wheels 1118 by a drive shaft. Although only a single motor is shown in FIG. 11A, in some implementations, there may be two or more motors with separate wheels coupled to separate motors. In some implementations, the motor or motors 1118 may be integrated into the wheels. In the series hybrid, the internal combustion engine does not directly drive the wheels.

In the parallel hybrid 1100C shown in FIG. 11C the internal combustion engine 1102 and traction electric motor 1116 are both mechanically coupled to the wheels. In the illustrated example, both the engine 1102 and electric motor 1116 are coupled by corresponding drive shafts 1120A, 1120B to a first differential 1122A that is mechanically coupled to a second differential 1122C that is coupled to the wheels 1118 by drive axels 1124. In the parallel hybrid, either the internal combustion engine 1102 or the electric motor 1116 or possibly both may selectively directly drive the wheels 1118. In the illustrated example the drive train is configured so that either the engine 1102 or the electric motor 1116 may drive the same pair of wheels. However, aspects of the present disclosure are not limited to such implementations. For example, the engine may drive one set of wheels, e.g., the rear wheels and the electric motor may be configured to drive another set of wheels, e.g., the front wheels, or vice versa. As with the electric vehicle of FIG. 11A and the series hybrid of FIG. 11B, electric motors may drive individual wheels 1118 directly and may even be integrated into the wheels themselves.

FIG. 1 shows an example of an electric vehicle 10. This electric vehicle 10 is provided with an electric drive motor 12 for traveling, and charging equipment 13 which are arranged at the rear part of a vehicle body 11, a CESM 14 arranged under the floor of the vehicle body 11, and the like. A heat exchanger unit 15 for air-conditioning is arranged at the front part of the vehicle body 11.

Front wheels 20 of the vehicle 10 are supported by the vehicle body 11 by means of front suspensions (not shown). Rear wheels 21 are supported by the vehicle body 11 by means of rear suspensions (not shown). An example of the rear suspension is a trailing arm type rear suspension.

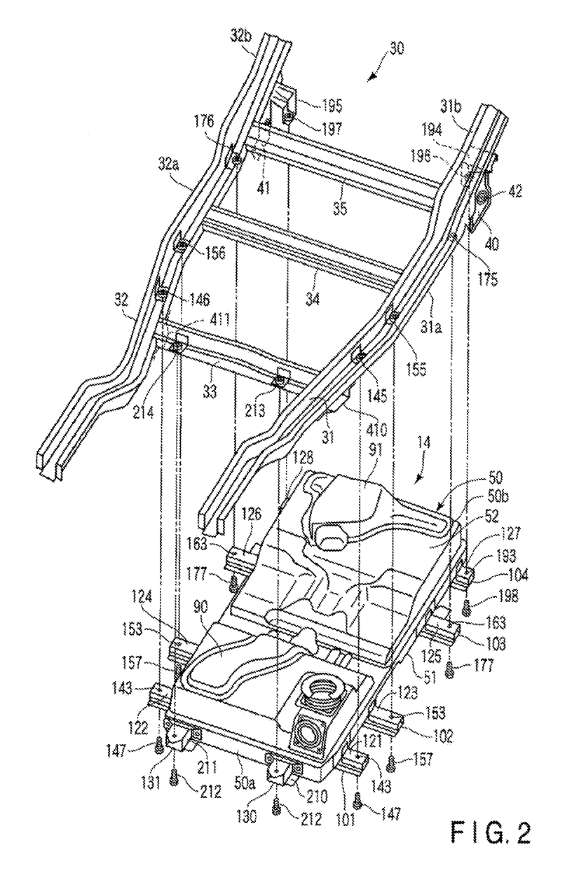

FIG. 2 shows a frame structure 30 constituting a lower skeletal structure of the vehicle body 11, and the CESM 14 to be attached to the frame structure 30.

The frame structure 30 includes a pair of right and left side members 31 and 32 extending in the back-and-forth direction of the vehicle body 11, and cross members 33, 34, and 35 extending in the width direction of the vehicle body 11. The cross members 33, 34, and 35 are fixed to predetermined positions of the side members 31 and 32 by welding. Each of the side members 31 and 32, and cross members 33, 34, and 35 is constituted of metal (for example, steel). That is, the side members 31 and 32 also function as an electromagnetic shield portion for intercepting electromagnetic waves from both the right and left sides of the CESM 14.

Suspension arm support brackets 40 and 41 are provided at the rear parts of the side members 31 and 32. Each of the suspension arm support brackets 40 and 41 is fixed to a predetermined position of each of the side members 31 and 32 by welding. Each of the suspension arm support brackets 40 and 41 is provided with an axis section 42. Front end parts of the trailing arms are attached to these axis sections 42.

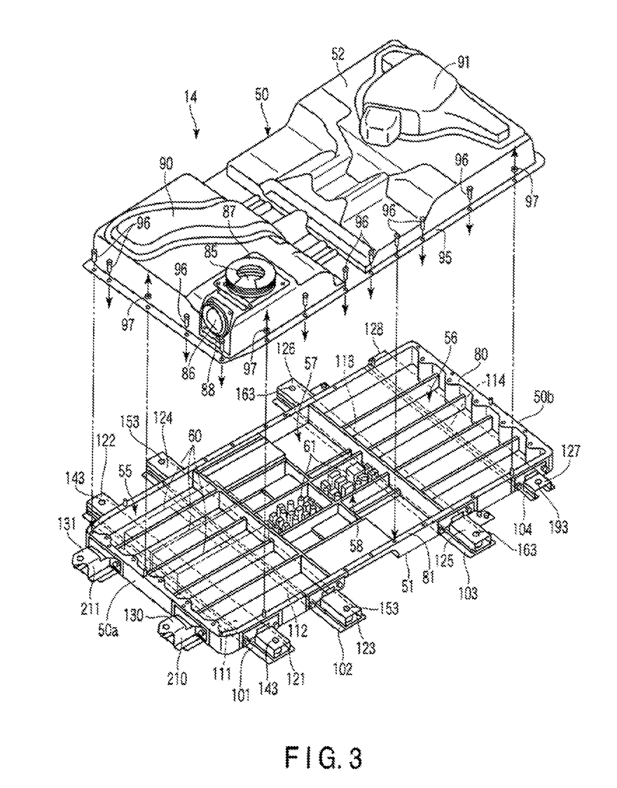

As shown in FIG. 3, the CESM 14 is provided with a CESM case 50. The CESM case 50 includes a tray member 51 positioned on the lower side, and a cover member 52 positioned on the upper side. A front CESM containing section 55 is formed at the front half part of the CESM case 50. A rear CESM containing section 56 is formed at the rear half part of the CESM case 50. A center CESM containing section 57, an electric circuit containing section 58, and the like are formed between the front CESM containing section 55 and rear CESM containing section 56.

A CESM 60 (only part thereof is shown by two-dot chain lines in FIG. 3) is contained in each of the CESM containing sections 55, 56, and 57. An example of the CESM 60 is a CESM formed by connecting, in series, a plurality of CESC each of which is constituted of a capacitive energy storage device (CESD). Wherein a CESD is constituted of one or more metacapacitors.

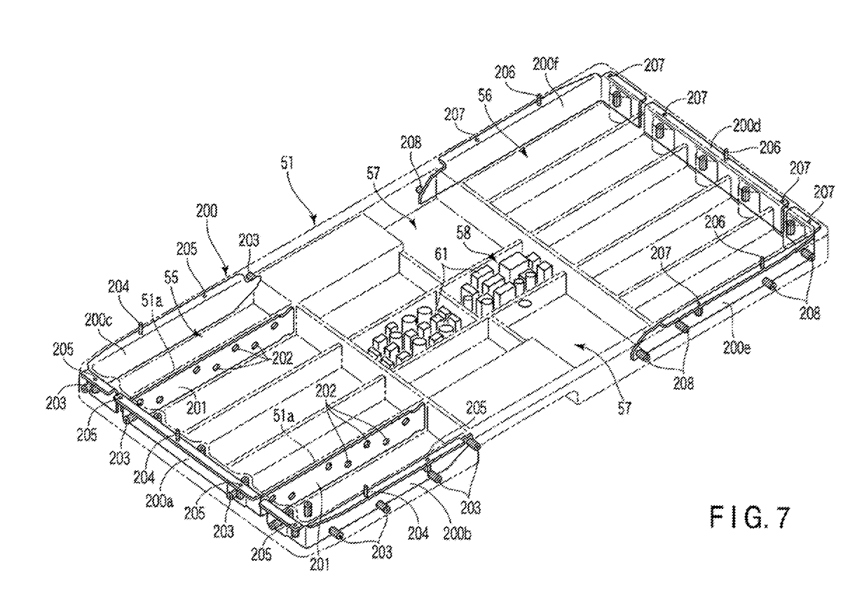

A monitor for detecting a state of the CESM 60, electric components 61 (part of them are schematically shown in FIGS. 3 and 7) and the like for managing control and the like are contained in the electric circuit containing section 58. The electric components 61 are electrically connected to the CESM 60. There is the possibility of the electric components 61 being adversely affected by an electromagnetic wave, and hence the electric components 61 are provided with an electromagnetic shield means to be described later.

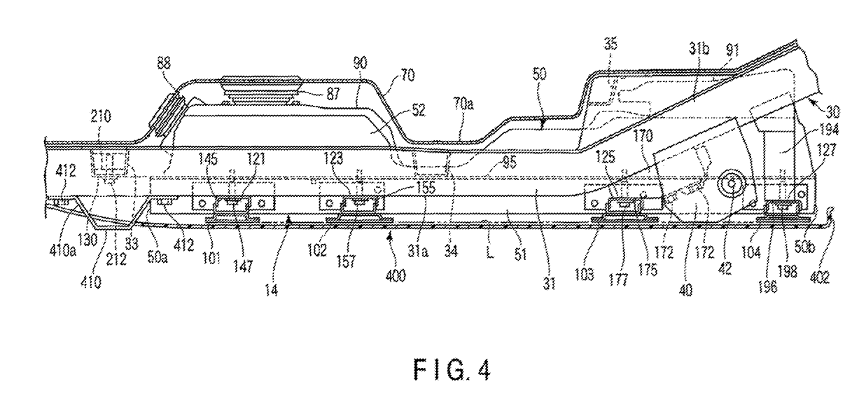

As shown in FIG. 4, the CESM 14 is arranged on the undersurface side of a floor panel 70 made of steel, and having an electromagnetic shielding effect. The upper side of the electric components 61 is magnetically shielded by the floor panel 70. That is, the floor panel 70 also functions as an upper electromagnetic shield portion for intercepting an electromagnetic wave from above the CESM 14. Further, the floor panel 70 also functions as a shield for preventing an electromagnetic wave generated from the CESM 14 from being directed upward. The floor panel 70 extends in the back-and-forth direction and width direction of the vehicle body 11 to constitute a floor part of the vehicle body 11.

The floor panel 70 is fixed to a predetermined position of the frame structure 30 including the side members 31 and 32 by welding. Front seats 71 (shown in FIG. 1) and rear seats 72 are arranged above the floor panel 70. The front CESM containing section 55 of the CESM 14 is arranged below the front seats 71. The rear CESM containing section 56 of the CESM 14 is arranged below the rear seats 72. The floor panel 70 includes a concave portion 70a. The concave portion 70a is formed between the front CESM containing section 55 and rear CESM containing section 56. This concave portion 70a is positioned in the vicinity of the feet of the occupants seated on the rear seats 72.

The tray member 51 is a molded article formed by inserting metal insert members 200 (shown in FIG. 7) for reinforcement in an integrally molded synthetic resin member. This tray member 51 is formed into a box-like shape opened at its top surface. The synthetic resin which is the material of the tray member 51 is reinforced by, for example, fibers. A cover fitting surface 80 (shown in FIG. 3) is formed at a peripheral edge part of the top surface of the tray member 51. The cover fitting surface 80 is continuous over the whole circumference of the tray member 51. A waterproof seal member 81 is provided on the cover fitting surface 80.

As shown in FIG. 7, the insert members 200 includes three metal plates 200a, 200b, and 200c positioned on the front side of the tray member 51, and three metal plates 200d, 200e, and 200f positioned on the rear side of the tray member 51. These metal plates 200a to 200f are constituted of a metallic material having an electromagnetic shielding effect, and large bending rigidity, for example, a steel sheet.

The metal plates 200a, 200b, and 200c which are positioned on the front side are embedded at positions corresponding to the front part, and right left sides of the front CESM containing section 55. A pair of right and left reinforcement plates 201 extending rearward is provided at both ends of the metal plate 200a on the center-front side. These reinforcement plates 201 are provided inside the resin constituting partition walls 51a of the tray member 51. These metal plates 200a , 200b , and 200c have a function of reinforcing the peripheral wall of the tray member 51. The metal plate 200a on the center-front side functions also as a front electromagnetic shield portion positioned on the vehicle-front side with respect to the electric components 61.

A plurality of holes 202 are formed in these reinforcement plates 201 in order to improve sticking of the plates 201 to the resin constituting the partition walls 51a. Further, each of the metal plates 200a , 200b , and 200c is provided with embedded nuts 203 protruding in the horizontal direction, an anchor bolt 204 protruding upward, and nut portions 205 each of which is provided with a threaded hole.

The metal plates 200d , 200e , and 200f on the rear side are embedded at positions corresponding to the rear part, and right left sides of the rear CESM containing section 56 of the tray member 51. An anchor bolt 206 protruding upward, and nut portions 207 each of which is provided with a threaded hole are provided on a top surface of each of the metal plates 200d, 200e, and 200f Embedded nuts 208 protruding in the horizontal direction are fixed to the pair of right and left metal plates 200e and 200f. These metal plates 200d, 200e, and 200f have a function of reinforcing the peripheral wall of the tray member 51.

The metal plate 200d on the center-rear side functions also as a rear electromagnetic shield portion positioned on the vehicle-rear side with respect to the electric components 61. It should be noted that each of a front wall and rear wall of the CESM case 50 may be provided with an electromagnetic shield member such as a metal mesh member in order to enhance the electromagnetic shielding effect at each of front and rear directions of the electric components 61. Each of right and left walls of the CESM case 50 may also be provided with an electromagnetic shield member such as a metal mesh member in order to enhance the electromagnetic shielding effect at each of right and left areas of the CESM case 50.

The cover member 52 is constituted of an integrally molded product of a synthetic resin reinforced by fibers. An opening 85 for service plug and cooling air introduction opening 86 are formed at a front part of the cover member 52. A bellows-like boot 87 is attached to the opening 85 for service plug. A bellows-like boot 88 is also attached to the cooling air introduction opening 86. A bypass flow path 90 for causing part of the cooling air to flow there through, cooling fan containing section 91, and the like are provided on the top surface of the cover member 52.

A flange portion 95 is formed at a peripheral edge part of the cover member 52. The flange portion 95 is continuous over the whole circumference of the cover member 52. A rear metal plate (not shown) made of metal serving as measures against a rear collision is arranged on the rear surface of the cover member 52. The rear metal plate is fixed to the tray member 51 together with the flange portion 95 of the cover member 52, and can function as a rear electromagnetic shield portion.

The flange portion 95 of the cover member 52 is placed on the cover fitting surface 80 of the tray member 51. Further, the tray member 51 and cover member 52 are fixed to each other through the seal member 81 in a watertight manner by means of bolts 96 and nuts 97 shown in FIG. 3.

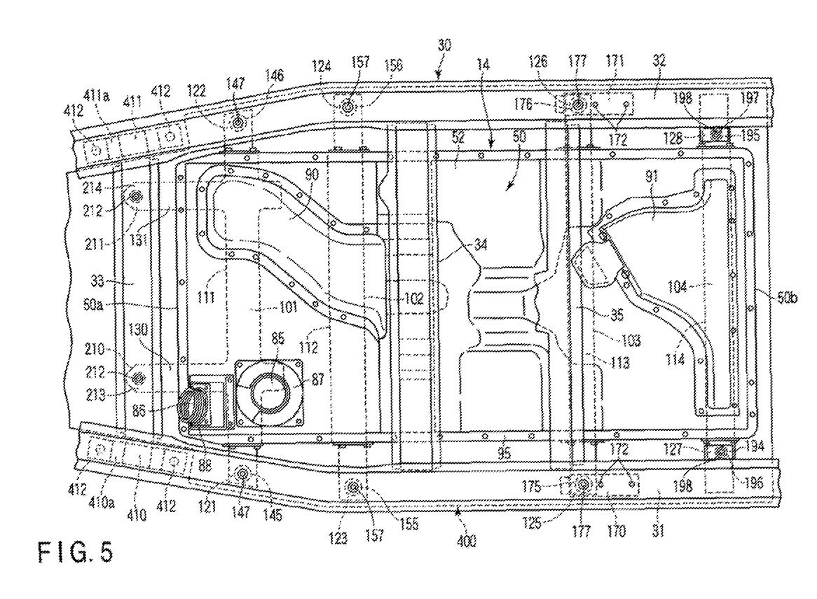

A plurality of (for example, four) beam members 101, 102, 103, and 104 are provided on the undersurface side of the tray member 51. As shown in FIGS. 3 and 5, the beam members 101, 102, 103, and 104 respectively include beam bodies 111, 112, 113, and 114 extending in the width direction of the vehicle body 11.

The first beam body 111 from the front is provided with joining portions 121 and 122 at both ends thereof. The second beam body 112 from the front is provided with joining portions 123 and 124 at both ends thereof. The third beam body 113 from the front is provided with joining portions 125 and 126 at both ends thereof. The fourth (rearmost) beam body 114 from the front is provided with joining portions 127 and 128 at both ends thereof. A pair of right and left front support members 130 and 131 is provided at a front end part of the CESM 14.

The beam members 101, 102, 103, and 104 are each provided with strength sufficient to support the weight of the CESM 14. Furthermore, these beam members are constituted of a metallic material (for example, a steel sheet) having an effect of intercepting an electromagnetic wave. That is, the beam members 101, 102, 103, and 104 function also as an under electromagnetic shield portion for intercepting an electromagnetic wave from below the CESM 14.

A bolt inserting hole 143 (shown in FIGS. 2 and 3) is formed in each of the joining portions 121 and 122 provided at both the ends of the first beam member 101 from the front. The bolt inserting hole 143 penetrates the joining portion 121 or 122 in the vertical direction. The side members 31 and 32 are provided with CESM fitting portions 145 and 146 at positions opposed to the joining portions 121 and 122. The CESM fitting portions 145 and 146 are provided with nut members. A bolt 147 (shown in FIGS. 2 and 4) is inserted into the bolt inserting hole 143 from below the joining portion 121 or 122. The bolt 147 is screwed into the nut member of the CESM fitting portion 145 or 146 to be fastened. As a result of this, the joining portions 121 and 122 of the first beam member 101 are fixed to the side members 31 and 32.

A bolt inserting hole 153 (shown in FIGS. 2 and 3) is formed in each of the joining portions 123 and 124 provided at both the ends of the second beam member 102 from the front. The bolt inserting hole 153 penetrates the joining portion 123 or 124 in the vertical direction. The side members 31 and 32 are provided with CESM fitting portions 155 and 156 at positions opposed to the joining portions 123 and 124. The CESM fitting portions 155 and 156 are provided with nut members. A bolt 157 (shown in FIGS. 2 and 4) is inserted into the bolt inserting hole 153 from below the joining portion 123 or 124. The bolt 157 is screwed into the nut member of the CESM fitting portion 155 or 156 to be fastened. As a result of this, the joining portions 123 and 124 of the second beam member 102 are fixed to the side members 31 and 32.

A bolt inserting hole 163 (shown in FIGS. 2 and 3) is formed in each of the joining portions 125 and 126 provided at both the ends of the third beam member 103 from the front. The bolt inserting hole 163 penetrates the joining portion 125 or 126 in the vertical direction. As shown in FIGS. 4 and 5, load transmission members 170 and 171 are fixed to the side members 31 and 32 by means of bolts 172. The load transmission members 170 and 171 are provided above the joining portions 125 and 126 of the third beam member 103 from the front. The one load transmission member 170 is welded to one suspension arm support bracket 40. The other load transmission member 171 is welded to the other suspension arm support bracket 41.

That is, the load transmission members 170 and 171 are joined to the side members 31 and 32, and suspension arm support brackets 40 and 41. These load transmission members 170 and 171 constitute part of the frame structure 30. The load transmission members 170 and 171 are provided with CESM fitting portions 175 and 176 including nut members.

A bolt 177 is inserted into the bolt inserting hole 163 from below the joining portion 125 or 126. The bolt 177 is screwed into the nut member of the CESM fitting portion 175 or 176 to be fastened. As a result of this, the joining portions 125 and 126 of the third beam member 103 are fixed to the side members 31 and 32 through the load transmission members 170 and 171.

A bolt inserting hole 193 (shown in FIGS. 2 and 3) is formed in each of the joining portions 127 and 128 of the fourth beam member 104 from the front. The bolt inserting hole 153 penetrates the joining portion 127 or 128 in the vertical direction. The side members 31 and 32 are provided with extension brackets 194 and 195 at positions opposed to the joining portions 127 and 128. The extension brackets 194 and 195 extend to positions beneath kick-up frame portions 31b and 32b of the side members 31 and 32. The extension brackets 194 and 195 constitute part of the frame structure 30. These extension brackets 194 and 195 are provided with CESM fitting portions 196 and 197 including nut members.

A bolt 198 (shown in FIGS. 2 and 4) is inserted into the bolt inserting hole 193 from below the joining portion 127 or 128. The bolt 198 is screwed into the nut member of the CESM fitting portion 196 or 197 to be fastened. As a result of this, the joining portions 127 and 128 of the fourth beam member 104 are fixed to the side members 31 and 32 through the extension brackets 194 and 195.

As shown in FIG. 4, undersurfaces of the beam members 101, 102, 103, and 104 are positioned on the same plane L extending in the horizontal direction along the flat undersurface of the tray member 51. The first and second beam members 101 and 102 are directly fixed to the CESM fitting portions 145, 146, 155, and 156 provided at the horizontal portions 31a and 32a of the side members 31 and 32.

The third and fourth beam members 103 and 104 are fixed to the CESM fitting portions 175, 176, 196, and 197 provided beneath the kick-up frame portions 31b and 32b of the side members 31 and 32. That is, the third and fourth beam members 103 and 104 are located at positions downwardly offset from the kick-up frame portions 31b and 32b. Accordingly, the third beam member 103 is fixed to the CESM fitting portions 175 and 176 through the load transmission members 170 and 171 each of which has a certain thickness in the vertical direction. The fourth beam member 104 is fixed to the CESM fitting portions 196 and 197 by means of the extension brackets 194 and 195 extending to the positions beneath the kick-up frame portions 31b and 32b.

The front support members 130 and 131 which are located at the front end of the CESM 14 protrude forward from the first beam member 101 from the front. The front support members 130 and 131 are joined to the beam member 101. As shown in FIG. 2, joining portions 210 and 211 provided to the front support members 130 and 131 are fixed to the CESM fitting portions 213 and 214 of the cross member 33 by means of bolts 212.

As described above, the beam members 101, 102, 103, and 104 of the electric vehicle of this embodiment are provided between the right and left side members 31 and 32. The side members 31 and 32 are joined to each other by the beam members 101, 102, 103, and 104. Thus, the beam members 101, 102, 103, and 104 of the CESM 14 can function as rigid members corresponding to the cross members.

Further, the load transmission members 170 and 171 are fixed to the suspension arm support brackets 40 and 41. The load in the transverse direction input to the suspension arm support brackets 40 and 41 is input to the beam member 103 through the load transmission members 170 and 171.

It is possible to enhance the rigidity of the parts around the suspension arm support brackets 40 and 41 by the beam member 103 even when a cross member is not arranged near the suspension arm support brackets 40 and 41. Accordingly, the steering stability and ride quality of the electric vehicle 10 are improved. In other words, it is possible to arrange part of the large-sized CESM 14 in a space between the pair of right and left suspension arm support brackets 40 and 41. As a result of this, it becomes possible to mount the large-sized CESM 14 on the electric vehicle, and prolong the travel distance of the electric vehicle.

As shown in FIGS. 1 and 4 to 7, an under cover 400 is arranged under the CESM 14. A top surface of the under cover 400 is opposed to the undersurfaces of the beam members 101, 102, 103, and 104. An example of a material of the under cover 400 is a synthetic resin reinforced with glass fibers. The under cover 400 is divided into, for example, a front half portion 400a and rear half portion 400b. By connecting the front half portion 400a and rear half portion 400b to each other, one under cover 400 is constituted. It should be noted that an under cover integral over the whole length may also be used.

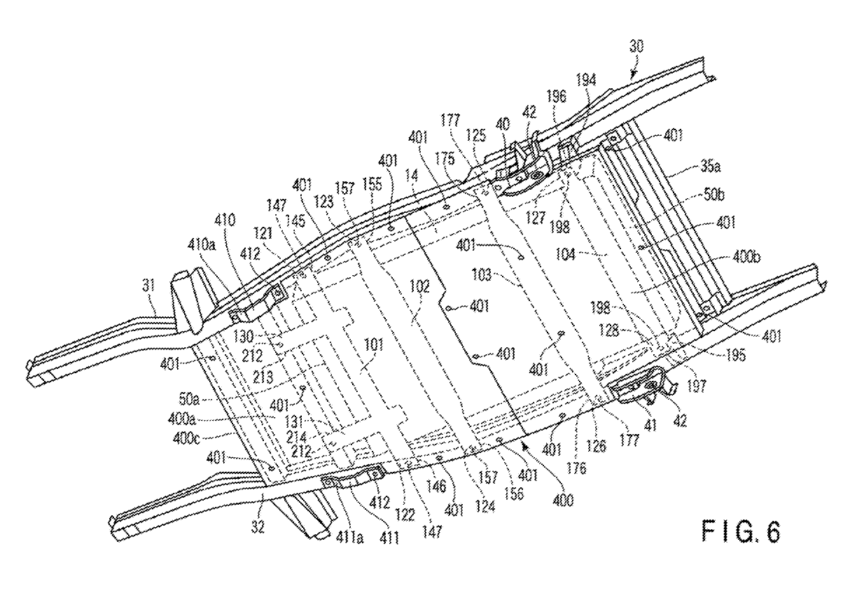

The CESM 14 is fixed to the frame structure 30 by means of the bolts 147, 157, 177, 198, and 212. Thereafter, the under cover 400 is fixed to at least part of the frame structure 30 and beam members 101, 102, 103, and 104 from below the vehicle body 11 by means of bolts 401 (shown in FIG. 6).

An overall length of the under cover 400 is larger than that of the CESM 14. That is, the under cover 400 has a length sufficient to cover from the front end 50 a to the rear end 50b of the CESM case 50. A width of the under cover 400 is larger than that of the CESM 14.

As shown in FIGS. 5 and 6, the under cover 400 is arranged over the pair of side members 31 and 32. A front portion of the under cover 400 is fixed to the front cross member 33a by means of the bolts 401. A central portion of the under cover 400 is fixed to the beam members 101, 102, 103, and 104, and tray member 51. A rear portion of the under cover 400 is fixed to brackets (not shown) provided on the rear cross member 35a (shown in FIG. 6) by means of bolts 401. The under cover 400 has an area sufficient to cover the whole undersurface of the CESM 14 when viewed from below the vehicle body 11.

The under cover 400 has a shape opened on the rear side. As a result of this, the electromagnetic shield on the rear side of the CESM 14 is constituted of the metal plates 200d, 200e, and 200f embedded in the rear portion of the tray member 51, and rear metal plate arranged on the rear surface of the cover member 52.

The bolts 147, 157, 177, 198, and 212 are covered with the under cover 400 from below. Thus, even if by any chance the bolts 147, 157, 177, 198, and 212 come off, the bolts fall onto the under cover 400. Thus, the occupant of this vehicle can recognize that a bolt has come off by the sound generated by the falling bolt striking the undercover 400, sound generated by the rolling bolt during the running, and the like.

Component dropping prevention walls 402 (shown in FIGS. 1 and 4) having a shape of an upward protrusion are formed at the peripheral edge portion of the under cover 400. It is recommended that the component dropping prevention walls 402 be provided at least at positions of the entire circumference of the under cover 400 enabling the walls 402 to prevent the bolt 147, 157, 177, 198, or 212 from rolling down from the under cover 400. A component such as the bolt or the like that has fallen onto the under cover 400 is retained inside the under cover 400 by the component dropping prevention walls 402, and hence the above component is prevented from falling onto the road.

As shown in FIG. 1, the under cover 400 is provided with a shield member 405 having an electromagnetic shielding effect. An example of the shield member 405 is a metal mesh member formed by braiding metallic wires into a reticular form.

This shield member 405 has a function of protecting the electric components 61 (shown in FIGS. 3 and 6) contained inside the CESM case 50 from an electromagnetic wave. It is particularly possible to prevent an electromagnetic wave generated from the motor 12 or the like from extending to the CESM case 50 from below the vehicle body 11 by means of the shield member 405 of the under cover 400. That is, the under cover 400 provided with the shield member 405 also functions as an under electromagnetic shield portion for intercepting an electromagnetic wave from below the CESM 14.

An electric component generating an electromagnetic wave is arranged between the floor panel 70 and the under cover 400. Above the floor panel 70, electronic equipment such as a radio, electronic clock, navigation system, and the like are arranged. In the electronic vehicle 10 of this embodiment, it is possible to prevent an influence of the electromagnetic wave generated from the electric component from extending to the electronic equipment by the electromagnetic shield.

It should be noted that the shield member 405 (schematically shown in FIG. 1) to be provided on the under cover 400 may be provided only on part of the under cover 400 in accordance with the intensity of the electromagnetic wave reaching the CESM case 50 from below the vehicle body 11, or the area to which the electromagnetic wave extends. Further, when the intensity of the electromagnetic wave is low to such an extent that there is practically no problem, the under cover 400 may not be provided with a shield member 405.

As shown in FIGS. 2, 5, and 6, the frame structure 30 is provided with protection members 410 and 411 functioning as a CESM protection means. In this embodiment, the CESM protection means is constituted of the protection members 410 and 411, and under cover 400.

The protection members 410 and 411 are attached to the undersurfaces of the side members 31 and 32 by means of bolts 412. The protection members 410 and 411 are attached to the front portions of the side members 31 and 32, i.e., the portions at which the distance between the side members 31 and 32 becomes smaller toward the front wheels 20. Thus, the protection members 410 and 411 are positioned inside the pair of right and left front wheels 20 when viewed from the front of the vehicle body 11. Furthermore, the under cover 400 extends rearward behind the protection members 410 and 411.

Inclined surfaces 410a and 411a are formed at front portions of the protection members 410 and 411. These inclined surfaces 410a nd 411a have a sled-like shape inclined to be higher from the front side of the side members 31 and 32 toward the rear side. A convex part of the road surface colliding against the protection members 410 and 411 from the front side of the vehicle body 11 while the vehicle is running is guided toward the rear of the protection members 410 and 411 along the inclined surfaces 410a and 411a.

The inclined surfaces 410a and 411a are positioned on the front side of the front end 50 a of the CESM case 50. The undersurfaces of the protection members 410 and 411 protrude downwardly from the side members 31 and 32. The undersurfaces of the protection members 410 and 411 are positioned lower than the undersurface of the front end 50a of the CESM case 50. Further, the undersurfaces of the protection members 410 and 411 protrude downwardly from the undersurface of the under cover 400.

In this way, the pair of right and left protection members 410 and 411 are fixed to the side members 31 and 32 having high rigidity. Further, the beam members 101, 102, 103, and 104 each having high rigidity are arranged behind the protection members 410 and 411 above the under cover 400.

When the vehicle is passing the large convex part such as a step or the like, it can be presumed, depending on the situation, for example, that the convex part of the road surface hits the under cover 400 at around the front end 400c thereof. In this case, the load of the collision is received by the cross member 33a arranged near the front end 400c of the under cover 400, and the convex part of the road surface is guided toward the rear side of the vehicle body 11 along the under cover 400. This makes it possible to prevent the convex part of the road surface from hitting the CESM 14.

Depending on the situation during the travel of the vehicle, it can be presumed that the convex part of the road surface collides against at least one of the protection members 410 and 411. In such a case, the inclined surfaces 410a and 411a of the protection members 410 and 411 run on the convex part of the road surface, thereby guiding the convex part toward the rear side of the vehicle body 11. In this case, the inclined surfaces 410a and 411a of the protection members 410 and 411 fulfill the function corresponding to a sled. That is, the convex part of the road surface that has hit the inclined surfaces 410a and 411a is guided toward the under cover 400. As a result of this, it is possible to prevent the convex part of the road surface from directly hitting the CESM 14.

The beam members 101, 102, 103, and 104 each having high rigidity are arranged above the under cover 400 from the front side of the vehicle body 11 toward the rear side thereof. These beam members 101, 102, 103, and 104 are arranged behind the protection members 410 and 411. This enables the under cover 400 to exert high strength against external force applied thereto from below. Thus, it is possible to guide the convex part of the road surface that has been brought into contact with the under cover 400 toward the rear side of the vehicle body 11 along the under cover 400. In this way, the CESM 14 can be prevented from being damaged. The under cover 400 has an effect of straightening the air current generated on the undersurface side of the vehicle body 11 while the vehicle is running, and can reduce the air resistance while the vehicle is running.

In the electric vehicle 10 of this embodiment, the CESM 14 is arranged below the floor panel 70 made of metal. The floor panel 70 functions as an upper electromagnetic shield portion. Further, the CESM 14 is arranged between the right and left side members 31 and 32 made of metal. That is, the side members 31 and 32 made of steel, having an effect of shielding the inside from an electromagnetic wave are arranged on both the right and left sides of the electric circuit containing section 58. These side members 31 and 32 function as electromagnetic shield portions on both the right and left sides of the CESM 14. Further, the cross members 33, 34, and 35 can also function as electromagnetic shield portions.

Furthermore, the beam members 101, 102, 103, and 104 made of metal, functioning as the under electromagnetic shield portion are present under the CESM case 50. Further, the metal plates 200 a and 200 d embedded in the resin constituting the CESM case 50 function as the front electromagnetic shield portion and rear electromagnetic shield portion, respectively.

Moreover, the under cover 400 provided with the electromagnetic shield means such as the shield member 405 is present under the CESM case 50. When the beam members 101 to 104 made of metal are present above the under cover 400, the beam members 101 to 104 exhibit an electromagnetic shielding effect. This makes it possible to reduce or omit the electromagnetic shield means to be provided on the under cover 400. That is, portions of the under cover 400 overlapping the beam members 101 to 104 when viewed from above the vehicle body 11 may not be provided with the shield member 405.

By providing the electromagnetic shield means described above, it is possible to prevent the electric components 61 inside the CESM case 50 from being adversely affected by an electromagnetic wave even when the paint for electromagnetic shield is not applied to the CESM case 50. Further, even if an electromagnetic wave is generated inside the CESM case 50, it is possible to prevent the electromagnetic wave from being radiated to the outside of the CESM case 50. According to the electromagnetic shield portions of this embodiment, it is possible not to arrange a conductive member for electromagnetic shielding on the inner surface of the CESM case 50 in a naked state. As a result of this, the conductive members for electromagnetic shielding do not give rise to a short circuit, which provides safety.

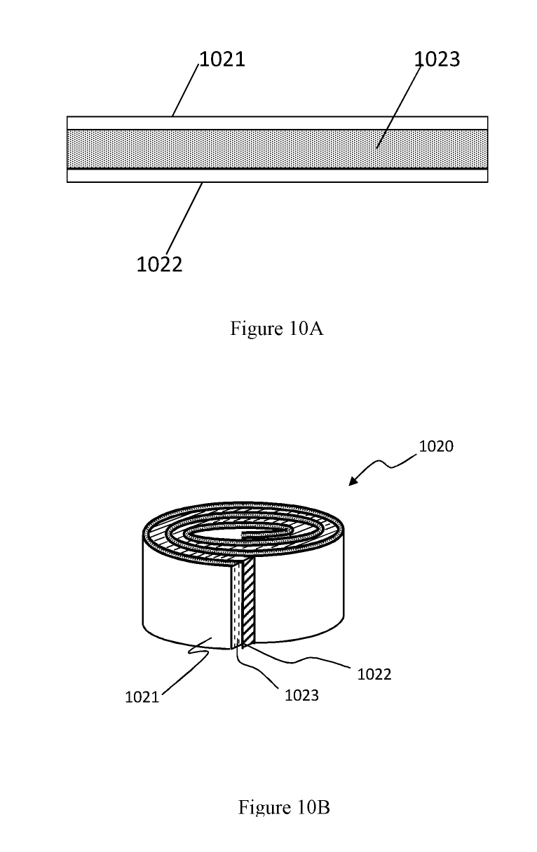

According to an aspect of the present disclosure a metacapacitor may be configured as shown in FIG. 10A. The metacapacitor comprises a first electrode 1021, a second electrode 1022, and a metadielectric layer 1023 disposed between said first and second electrodes. The electrodes 1021 and 1022 may be made of a metal, such as copper, zinc, or aluminum or other conductive material and are generally planar in shape.

The electrodes 1021, 1022 may be flat and planar and positioned parallel to each other. Alternatively, the electrodes may be planar and parallel, but not necessarily flat, e.g., they may be coiled, rolled, bent, folded, or otherwise shaped to reduce the overall form factor of the capacitor. It is also possible for the electrodes to be non-flat, non-planar, or non-parallel or some combination of two or more of these. By way of example and not by way of limitation, a spacing d between the electrodes 1021, 1022, which may correspond to the thickness of the Composite Dielectric Film layer 1023, may range from about 100 nm to about 10,000 μm. As noted in Equation (2) below, the maximum voltage V_{bd} between the electrodes 1021, 1022 is approximately the product of the breakdown field E_{bd} and the electrode spacing d.

V_{bd}=E_{bd}d (2)

For example, if, E_{bd}=0.1 V/nm and the spacing d between the electrodes 1, 2 is 10,000 microns (100,000 nm), the maximum voltage V_{bd} would be 100,000 volts.

The electrodes 1021, 1022 may have the same shape as each other, the same dimensions, and the same area A. By way of example, and not by way of limitation, the area A of each electrode 1021, 1022 may range from about 0.01 m² to about 1000 m². By way of example and not by way of limitation, for rolled capacitors, the electrodes may be up to, e.g., 1000 m long and 1 m wide.

These ranges are non-limiting. Other ranges of the electrode spacing d and area A are within the scope of the aspects of the present disclosure.

If the spacing d is small compared to the characteristic linear dimensions of electrodes (e.g., length and/or width), the capacitance C of the capacitor may be approximated by the formula:

C=κϵₒA/d (3)

where ϵₒ is the permittivity of free space (8.85×10⁻¹² Coulombs²/(Newton·meter²)) and κ is the dielectric constant of the dielectric layer. The energy storage capacity U of the capacitor may be approximated as:

U=½CV_{bd}² (4)

which may be rewritten using equations (2) and (3) as:

U=½κϵₒAE_{bd}²d (5)

The energy storage capacity U is determined by the dielectric constant κ, the area A, and the breakdown field E_{bd}. By appropriate engineering, a capacitor or capacitor bank may be designed to have any desired energy storage capacity U. By way of example, and not by way of limitation, given the above ranges for the dielectric constant K, electrode area A, and breakdown field E_{bd} a capacitor in accordance with aspects of the present disclosure may have an energy storage capacity U ranging from about 500 Joules to about 2×10¹⁶ Joules.

For a dielectric constant κ ranging, e.g., from about 100 to about 1,000,000 and constant breakdown field E_{bd} between, e.g., about 0.1 and 0.5 V/nm, a capacitor of the type described herein may have a specific energy capacity per unit mass ranging from about 10 W·h/kg up to about 100,000 W·h/kg, though implementations are not so limited.

Aspects of the present disclosure include the use of metacapacitors that are coiled, e.g., as depicted in FIG. 10B. In this example, a metacapacitor 1020 comprises a first electrode 1021, a second electrode 1022, and a metadielectric material layer 1023 of one or more of the types described hereinabove disposed between said first and second electrodes. The electrodes 1021, 1022 may be made of a metal, such as copper, zinc, or aluminum or other conductive material and are generally planar in shape. In one implementation, the electrodes and metadielectric material layer 1023 are in the form of long strips of material that are sandwiched together and wound into a coil along with an insulating material, e.g., a plastic film such as polypropylene or polyester to prevent electrical shorting between the electrodes 1021, 1022. Examples of such coiled capacitor energy storage devices are described in detail in commonly-assigned U.S. patent application Ser. No. 14/752,600, filed Jun. 26, 2015, which has been published as U.S. Patent Application Publication Number 2016/0379757, the entire contents of which are incorporated herein by reference.

In the above embodiment, an electric vehicle in which a motor for running is mounted on the rear part of the vehicle body has been described. However, the present invention can also be applied to an electric vehicle in which a motor for running is mounted on the front part of the vehicle body. Further, in carrying out the present invention, it goes without saying that the structures and arrangements of the constituent elements of the present invention such as the side members, floor panel, motor, CESM, front electromagnetic shield portion, rear electromagnetic shield portion, under electromagnetic shield portion, and the like can be appropriately modified and implemented.

As further example of a viable capacitor for vehicle applications, a coiled capacitor of dielectric film as described above with a mass of 306 grams would have 1001 Wh. Incorporated into a can capacitor it would be able to provide 40 amps in the range 200 V through 1000 V and a mass of 593 due to power electronics. This provides 40 A*200 V=8000 W, or 8 kW with 16 kW/kg and 1.7 kWh/kg power and energy densities. A capacitor power module with 10 or 5 such capacitors which are connected in parallel to a common cathode and common anode and wherein the common cathode and common anode have power electronics with a 40 amp rating. The voltage of each capacitor is 400 volts and the current charge discharge rate is nominally 20 amps and can be 0 to 40 amps. The nominal power of the module with 5 capacitors is 5*20 A*400 V=40 kW and for the 10 capacitor module is 10*20 A*400 V=80 kW. The high power rating enables rapid charge and discharge for accelerating and decelerating an electric vehicle. While a typical personal electric vehicle would theoretically require ˜1215 kg of battery for acceleration, it would only require 36.7 kg of supercapacitor or 17.7 kg of metacapacitor in addition to gasoline engine and fuel tank to build a hybrid. Similarly, a typical personal electric vehicle would theoretically require ˜444 kg of battery to meet range requirements, it would only require 58.8 kg of metacapacitor or a giant 13889 kg of supercapacitor to store energy equivalent to the battery pack.

Table 5 below compares energy density and power density for supercapacitors, lithium ion batteries and a metacapacitor with ϵ=50,000.

TABLE 5

| | | Lithium Ion | Metacapacitor |
|---|---|---|---|
| | Supercapacitor - | NCR18650A | with Epsilon |
| | 3.0 V/3000 F | Battery | 50,000 |
| Energy Density | 7.2 | 225 | 1700 |
| [Wh/kg] | | | |
| Power Density | 7.7 | 0.233 | 16 |
| [kW/kg] | | | |

Table 6 below summarizes the above advantages of metacapactor-based electric vehicles compared to those based on lithium battery or supercapacitor.

TABLE 6

| | Example Vehicle | | |
|---|---|---|---|
| | Energy | Performance | |
| | Storage | Long Range | |
| | Spec | EV | Hybrid EV |
| Curb weight [lbs] | | 4600 | 3580 |
| Target Speed [mph] | | 40 | 60 |
| Time [seconds] | | 2.8 | 7 |
| Drag area [Cd × Ft2] | | 6.2 | 8 |
| Drivetrain power loss [%] | | 15 | 15 |
| Battery Energy [Wh] | | 100000 | 60000 |
| Supercapacitors | | | |
| Required Kg for power | 7.7 | 36.7 | 26.8 |
| Required Kg for | 7.2 | 15.3 | 27.9 |
| acceleration energy | | | |
| Required Kg for range | 7.2 | 13888.9 | 8333.3 |
| energy | | | |
| Batteries | | | |
| Required Kg for power | 0.233 | 1214.8 | 888.1 |
| Required Kg for | 225 | 0.5 | 0.9 |
| acceleration energy | | | |
| Required Kg for range | 225 | 444.4 | 266.7 |
| energy | | | |
| Metacapacitors | | | |
| Required Kg for power | 16 | 17.7 | 12.9 |
| Required Kg for | 1700 | 0.1 | 0.1 |
| acceleration energy | | | |
| Required Kg for range | 1700 | 58.8 | 35.3 |
| energy | | | |

Another advantage to the use of metacapacitors is greater efficiency in regenerative braking. Regenerative braking is a system in which wasted kinetic energy that must be dissipated in order to slow a vehicle down is reclaimed for later use. Vehicular travel often involves periods of acceleration and deceleration due to uneven terrain, speed laws, or the avoidance of obstacles. This results in energy used to accelerate a vehicle being unused when deceleration is necessary. Deceleration is achieved by physically interfering with the motion of the axil rods transferring energy to the wheels. Regenerative braking takes advantage of this physical interaction to channel mechanical energy back into the electrical energy storage, traditionally a battery. With metacapacitors however, there is lower energy transfer losses as mechanical energy need not be translated into chemical energy and the metacapacitor is capable of accepting larger amounts of energy in a short time than battery systems.

The electric vehicle may have a plurality of wheels and a plurality of traction motors to which each are individually connected to a dedicated capacitor power module. Alternatively, in some embodiments, a plurality of traction motors may be electrically connected to a single capacitor power module. Another embodiment is where a single traction motor may be electrically connected to a single capacitor power module. In yet another embodiment, a plurality of traction motors may be connected to a system of capacitor power modules that are connected in parallel to a common cathode and anode that are themselves part of a power electronics circuit capable of managing discharge and charge rates; and monitoring and relaying temperature of the individual capacitor power modules, state of charge of the individual capacitor power modules to the electric vehicle's power management system.

Another aspect of the disclosure is a system of capacitor energy storage modules as commonly described in U.S. patent application Ser. No. 15/043,315 (Attorney Docket Number CSI-024), wherein one embodiment one or more traction motors are directly electrically connected to said system of capacitor energy storage modules and connected in parallel with one or more capacitor power modules. In another embodiment, the system of capacitor energy storage modules are directly electrically connected in series with one or more capacitor power modules, and the one or more capacitor power modules are electrically connected to one or more of the electric motors.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

While the above is a complete description of the preferred embodiment of the present invention, it is possible to use various alternatives, modifications and equivalents. Therefore, the scope of the present invention should be determined not with reference to the above description but should, instead, be determined with reference to the appended claims, along with their full scope of equivalents. Any feature described herein, whether preferred or not, may be combined with any other feature described herein, whether preferred or not. In the claims that follow, the indefinite article “A”, or “An” refers to a quantity of one or more of the item following the article, except where expressly stated otherwise. As used herein, in a listing of elements in the alternative, the word “or” is used in the logical inclusive sense, e.g., “X or Y” covers X alone, Y alone, or both X and Y together, except where expressly stated otherwise. Two or more elements listed as alternatives may be combined together. The appended claims are not to be interpreted as including means-plus-function limitations, unless such a limitation is explicitly recited in a given claim using the phrase “means for.”

What is claimed is:

1. An electric vehicle, comprising:
an electric control unit controlling all operations of said electric vehicle;
an energy storage unit having one or more energy devices based on metacapacitors which provide needed electric energy and power of said electric vehicle;
a DC-DC converter in communication with the electric control unit to convert the voltage of the metacapacitors to the input voltage required by the motor driver;
a motor driver receiving control signal from said electric control unit to convert the energy from said DC-DC converter then provide to motor in a fashion that makes the motor turn with power;
a motor converting the electric energy into mechanical energy to drive the wheels;
provisions to make all electric energy converter systems bi-directional so that acceleration will cause the energy in the energy storage unit to decrease and deceleration will cause the amount of energy stored in the system to increase; and
a plurality of wheels which are driven by said motor.

2. The electric vehicle of claim 1, wherein the energy storage unit comprises:
one or more capacitor energy storage cells(CESC) having metacapacitor group (20), a DC-DC converter (3), a control circuit (4);
a first general electrode and second general electrode connecting with the output terminal of said DC-DC converter (3), therein
said metacapacitor group (20) having more than two parallelly connected metacapacitors, the input terminal of said DC-DC converter (3) respectively connecting with electrodes of said parallelly connected two or more metacapacitors, the output terminal connecting with said first general electrode and second general electrode, said DC-DC converter converting the voltage of said parallelly connected two or more metacapacitors, according to the control signals from said electronic control unit,
control circuit (4) receiving control signals from said electronic control unit, controlling said DC-DC converter (3).

3. The electric vehicle of claim 1, wherein the energy storage unit comprises:
one or more capacitor energy storage cells(CESC) having metacapacitor group (20), a DC-DC converter (3), a control circuit (4);
a first general electrode and second general electrode connecting with the output terminal of said DC-DC converter (3), therein
said metacapacitor group (20) having a metacapacitor,
the input terminal of said DC-DC converter (3) connecting with electrode of said metacapacitor, the output terminal connecting with said first general electrode and second general electrode, said DC-DC converter converting the voltage of said metacapacitor,
according to the control signals from said electronic control unit,
control circuit (4) receiving control signals from said electronic control unit, controlling said DC-DC converter (3).

4. The electric vehicle of claim 1, wherein the energy storage unit comprises:
one or more capacitor energy storage cells (CESC) having metacapacitor group (20) that includes two or more parallelly connected metacapacitors;
a DC-DC converter (3) whose input terminal connecting with the electrode of said metacapacitor, output terminal connecting with first general electrode and second general electrode, said DC-DC converter converting the voltage of said two or more parallelly connected metacapacitors, according to the control signals from said electronic control unit,
a first general electrode and a second general electrode connecting with the output terminal of said DC-DC converter (3);
a control circuit (4) receiving control signals from said electronic control unit, controlling said DC-DC converter (3).

5. A series hybrid electric vehicle, comprising:
an electric control unit controlling all operations of said electric vehicle;
an energy storage unit having one or more energy devices based on metacapacitors which provide needed electric energy and power of said electric vehicle;
a DC-DC converter in communication with the electric control unit to convert the voltage of the metacapacitors to the input voltage required by the motor driver,;
a motor driver receiving control signal from said electric control unit to convert the energy from said DC-DC converter then provide to motor in a fashion that makes the motor turn with power;
a motor converting the electric energy into mechanical energy to drive the wheels;
an engine generating power by burning fuel;
a generator, accepting mechanical power from said engine and converting to electrical power to charge said energy storage unit;
provisions to make all electric energy converter systems bi-directional so that acceleration will cause the amount of energy stored in the energy storage unit to decrease and deceleration will cause the amount of energy stored in the energy storage unit to increase;
a plurality of wheels which are driven by said motor.

6. The series hybrid electric vehicle of claim 5, wherein the energy storage unit comprises:
one or more capacitor energy storage cells(CESC) having metacapacitor group (20), a DC-DC converter (3), a control circuit (4);
a first general electrode and second general electrode connecting with the output terminal of said DC-DC converter (3), therein
said metacapacitor group (20) having more than two parallelly connected metacapacitors,
the input terminal of said DC-DC converter (3) respectively connecting with electrodes of said parallelly connected two or more metacapacitors, the output terminal connecting with said first general electrode and second general electrode, said DC-DC converter converting the voltage of said parallelly connected two or more metacapacitors,
according to the control signals from said electronic control unit,
control circuit (4) receiving control signals from said electronic control unit, controlling said DC-DC converter (3).

7. The series hybrid electric vehicle of claim 5, wherein the energy storage unit comprises:
one or more capacitor energy storage cells(CESC) having metacapacitor group (20), a DC-DC converter (3), a control circuit (4);
a first general electrode and second general electrode connecting with the output terminal of said DC-DC converter (3), therein
said metacapacitor group (20) having a metacapacitor,
the input terminal of said DC-DC converter (3) connecting with electrode of said metacapacitor, the output terminal connecting with said first general electrode and second general electrode, said DC-DC converter converting the voltage of said metacapacitor,
according to the control signals from said electronic control unit,
control circuit (4) receiving control signals from said electronic control unit, controlling said DC-DC converter (3).

8. The series hybrid electric vehicle of claim 5, wherein the energy storage unit comprises:
one or more capacitor energy storage cells (CESC) having metacapacitor group (20) that includes two or more parallelly connected metacapacitors;
a DC-DC converter (3) whose input terminal connecting with the electrode of said metacapacitor, output terminal connecting with first general electrode and second general electrode, said DC-DC converter converting the voltage of said two or more parallelly connected metacapacitors, according to the control signals from said electronic control unit,
a first general electrode and a second general electrode connecting with the output terminal of said DC-DC converter (3);
a control circuit (4) receiving control signals from said electronic control unit, controlling said DC-DC converter (3).

9. A parallel hybrid electric vehicle, comprising:
an electric control unit controlling all operations of said electric vehicle;
an energy storage unit having one or more energy devices based on metacapacitors which provide needed electric energy and power of said electric vehicle;
a DC-DC converter in communication with the electric control unit to convert the voltage of the metacapacitors to the input voltage required by the motor driver;
a motor driver receiving control signal from said electric control unit to convert the energy from said DC-DC converter then provide to motor in a fashion that makes the motor turn with power;
a motor converting the electric energy into mechanical energy to drive the wheels;
an engine generating power by burning fuel;
a generator, accepting mechanical power from said engine and converting to electrical power to charge said energy storage unit; a drive train connected to the engine to convert some or all of the engine power to drive one or more wheels of a plurality wheels;
provisions to make all electric energy converter systems bi-directional so that acceleration will cause the amount of energy stored in the energy storage unit to decrease and deceleration will cause the amount of energy stored in the energy storage unit to increase;
wherein the plurality of wheels is configured to be selectively driven by said motor or said engine.

10. The parallel hybrid electric vehicle of claim 9, wherein the energy storage unit comprises:
one or more capacitor energy storage cells(CESC) having metacapacitor group (20), a DC-DC converter (3), a control circuit (4);
a first general electrode and second general electrode connecting with the output terminal of said DC-DC converter (3), therein
said metacapacitor group (20) having more than two parallelly connected metacapacitors, the input terminal of said DC-DC converter (3) respectively connecting with electrodes of said parallelly connected two or more metacapacitors, the output terminal connecting with said first general electrode and second general electrode, said DC-DC converter converting the voltage of said parallelly connected two or more metacapacitors,
according to the control signals from said electronic control unit,
control circuit (4) receiving control signals from said electronic control unit, controlling said DC-DC converter (3).

11. The parallel hybrid electric vehicle of claim 9, wherein the energy storage unit comprises:
one or more capacitor energy storage cells(CESC) having metacapacitor group (20), a DC-DC converter (3), a control circuit (4);
a first general electrode and second general electrode connecting with the output terminal of said DC-DC converter (3), therein
said metacapacitor group (20) having a metacapacitor,
the input terminal of said DC-DC converter (3) connecting with electrode of said metacapacitor, the output terminal connecting with said first general electrode and second general electrode, said DC-DC converter converting the voltage of said metacapacitor,
according to the control signals from said electronic control unit,
control circuit (4) receiving control signals from said electronic control unit, controlling said DC-DC converter (3).

12. The parallel hybrid electric vehicle of claim 9, wherein the energy storage unit comprises:
one or more capacitor energy storage cells (CESC) having metacapacitor group (20) that includes two or more parallelly connected metacapacitors;
a DC-DC converter (3) whose input terminal connecting with the electrode of said metacapacitor, output terminal connecting with first general electrode and second general electrode, said DC-DC converter converting the voltage of said two or more parallelly connected metacapacitors, according to the control signals from said electronic control unit,
a first general electrode and a second general electrode connecting with the output terminal of said DC-DC converter (3);
a control circuit (4) receiving control signals from said electronic control unit, controlling said DC-DC converter (3).
